(19) United States
(12) Patent Application Publication
(10) Pub. No.: US 2024/0252097 A1
(43) Pub. Date: Aug. 1, 2024

(54) ELECTROMYOGRAPHY SYSTEM FOR MEDICAL USE

(71) Applicant: STB INN AB, Träslövsläge (SE)

(72) Inventors: ANDREAS JONSSON, Träslövsläge (SE); JOHANNA HENRIKSSON WENNSTRÖM, Träslövsläge (SE); SIGNE SVENSSON, Träslövsläge (SE)

(73) Assignee: STB INN AB, Träslövsläge (SE)

(21) Appl. No.: 18/566,580

(22) PCT Filed: Jun. 1, 2022
(86) PCT No.: PCT/EP2022/064974

§ 371 Date: Dec. 1, 2023

(30) Foreign Application Priority Data
Jun. 3, 2021 (GB) 2107978.5

(51) Int. Cl. A61B 5/395; A61B 5/00; A61B 5/259; A61B 5/296

(52) U.S. Cl. CPC A61B 5/395; A61B 5/259; A61B 5/296; A61B 5/6823; A61B 5/7225; A61B 2562/0247; A61B 2562/043

(57) ABSTRACT

A surface electromyography system, sensors for use in a surface electromyography system, and methods for use thereof. The surface electromyography system comprises a plurality of sensors for detecting muscular electrical activity, and an output unit coupleable to the plurality of sensors, the output unit operable to provide outputs representing the outputs of each of the plurality of sensors. The sensors of the plurality of sensors each comprise a pair of electrodes and are operable to provide an output representing the potential difference between the pair of electrodes.

The technology described herein relates to an electromyography system for medical use, and in particular to a system for surface electromyography.

Electromyography (EMG) is a diagnostic technique that measures muscle activity by detecting the electrical activity of skeletal muscles. The electrical activity can be analysed to detect medical abnormalities. There are two forms of electromyography, intramuscular electromyography and surface electromyography (sEMG). Intramuscular electromyography involves the insertion of electrodes into muscles to detect the electrical activity of the muscle. In contrast, surface electromyography detects muscle activity from the surface of the skin above the muscle.

Surface electromyography is limited in that the detection of electrical activity is restricted to superficial muscles, and that the measurements are influenced by the depth of the subcutaneous tissue at the site of the recording. In addition, surface electromyography cannot reliably discriminate between the discharges of adjacent muscles.

Despite these limitations, the use of surface electromyography for rehabilitation and medical research is beneficial due to the less invasive nature of surface electromyography in comparison to intramuscular electromyography. For example, previous research has indicated that measurements of abdominal activity using surface electromyography may enable the detection of peritonitis.

The Applicants have therefore recognised that there is a need for improvements in the design and use of systems for electromyography, and in particular in systems for surface electromyography.

According to a first aspect of the technology described herein, there is provided a surface electromyography system for medical use, the system comprising a plurality of sensors for detecting muscular electrical activity and an output unit coupled to the plurality of sensors. Each sensor comprises a pair of electrodes and is operable to provide an output representing the potential difference between the pair of electrodes. The output unit is operable to provide outputs representing the outputs of each of the plurality of sensors.

The technology described herein also extends to performing tests to measure muscular electrical activity over a region of the surface of a subject's skin using the system of the technology described herein. Thus, according to a second aspect of the technology described herein, there is provided a method of measuring muscular electrical activity over a region of the surface of a subject's skin using the system of the technology described herein, the method comprising attaching the plurality of sensors to the subject's skin such that the pairs of electrodes are in contact with the subject's skin, detecting a potential difference between the pairs of electrodes, and providing an output representative of the potential difference detected between the pairs of electrodes.

The technology described herein further extends to the use of the system or method of the technology described herein to measure muscular electrical activity over a region of the surface of a subject's skin. Thus, a third aspect of the technology described herein comprises the use of the surface electromyography system of the technology described herein for measuring muscular electrical activity over a region of the surface of a subject's skin.

The technology described herein provides a method and apparatus for detecting and analysing muscular electrical signals that are medically useful or that could be used as an aid for forming a medical diagnosis. For example, the apparatus may be used to detect the electrical activity of abdominal muscles to aid in the diagnosis of peritonitis.

In contrast to existing electromyography systems, the technology described herein enables the simultaneous detection of electrical activity in multiple distinct regions using a plurality of sensors.

The Applicants have realised that the simultaneous use of multiple sensors for detecting muscular electrical activity provides several advantages over existing electromyography systems and methods. For example, many diagnostic methods utilising electromyography require measurements to be taken from several different regions. In existing systems, this requires relocating a single sensor several times over the course of each evaluation. This need to relocate the sensor is a source or error for the measurements as, for example, the movement of the patient during the relocation process may impair signal quality. In addition, by allowing measurements of the electrical activity in each region to be taken simultaneously, the technology described herein enables the measurements in each region to be directly compared to one another in real time. This can assist in, for example, detecting an incorrect or non-optimal placement of one or more of the sensors. The simultaneous use of the plurality of sensors provide further advantages relating to the improved accuracy of the measurements. For example, the likelihood of false positive or negative results is reduced by the use of the additional sensors.

In an embodiment, the system comprises four sensors. However, the system may comprise any number of sensors greater than one, including but not limited to 2, 3, 4, 5 or more sensors. The number of sensors may be selected based on the requirements of the particularities of the electromyography test.

As mentioned above, the present system enables the simultaneous detection of electrical activity in multiple distinct regions using a plurality of sensors. The sensors of the plurality of sensors are physically separate from one another, such that the position of any sensor of the plurality of sensors does not physically depend on (affect) the position of any other sensor of the plurality of sensors. Thus, the plurality of sensors are positionable independently (and simultaneously) at different positions on the skin of a subject (patient).

In an embodiment, the plurality of sensors are positionable such that at least one sensor of the plurality of sensors is positioned for measuring electrical activity of an external oblique muscle of the subject, and at least one (other) sensor of the plurality of sensors is simultaneously positioned for measuring electrical activity of an internal oblique muscle of the subject.

In an embodiment, the sensors of the plurality of sensors are (simultaneously) positionable at different locations on a subject's abdomen. In an embodiment, the plurality of sensors comprises four sensors (simultaneously) positionable in different quadrants of a subject's abdomen. In an embodiment, the sensors of the plurality of sensors are positionable such that a (different) sensor is positioned for simultaneously measuring electrical activity of each of: a left hand external oblique muscle, a right hand external oblique muscle, a left hand internal oblique muscle, and a right hand internal oblique muscle of a subject.

Each of the plurality of sensors comprises a pair of electrodes configured to detect the electrical potential between the electrodes when placed on the skin of a subject. The electrode pairs are in an embodiment arranged in a single differential (SD) configuration. The electrodes of the electrode pair will be separated by an inter-electrode distance (IED), the IED being the distance between the respective centres of the two electrodes. In an embodiment, the IED of each of the electrode pairs is between 3 mm and 50 mm, and further in an embodiment between 3 mm and 40 mm, 3 mm and 20 mm or 3 mm and 5 mm. The Applicant has found that this range of IEDs provides suitable sensors for detecting signals that are of interest for surface electromyography. Alternatively, a separation greater than 50 mm or a separation of between e.g. 15 mm and 40 mm, 15 mm and 30 mm, or 15 mm and 25 mm may be used. In an embodiment, a separation (IED) of 24 mm is used. For example, an IED greater than 50 mm may be suitable for use with subjects with a large amount of subcutaneous tissue at the sensing locations.

The Applicant has recognised that the quality of a sEMG signal greatly depends on the IED, as a larger IED results in a greater level of distortion of the sEMG signal. Typically, an IED greater than 10 mm (or, if less, greater than ¼ of the total muscle fibre length of the muscle being evaluated) will result in distortions of the sEMG signal. However, with increasing muscle depth, the sEMG amplitude at the skin decreases. This means that the amplitude of sEMG signals are influenced by, for example, the depth of the subcutaneous tissue at the site of the sensor, which can be highly variable depending on the weight (or Body Mass Index, “BMI”) of a patient. For example, a higher weight (or BMI) may result in relatively weaker signals. The Applicants have recognised that the amplitude of the sEMG signal may be increased by increasing the IED, at the cost of increasing the interference from neighbouring muscles and increasing the risk of cross talk and compromising the spectral content of the sEMG signal.

The sensors may be configured to have a (permanently) fixed IED. However, in an embodiment, the IED of the sensors can be varied. In an embodiment, the IED can be adjusted to any length between 3 mm and 50 mm, 3 mm and 40 mm, 3 mm and 20 mm, or 3 mm and 5 mm. For example, the sensors may have movable electrodes, such that the IED of the electrode pair is variable. The position of the electrodes is in an embodiment fixable, such that the electrodes can be securely positioned and thereby maintain a constant IED when the sensor is in use. Alternatively, the position of the electrodes may be fixed (not moveable), but the electrodes are removable and replaceable with electrodes of a different size (and thus having a different IED). Thus, in embodiments, the plurality of sensors are configured to receive various (plural different) sizes of electrode (such that the IED of the electrodes can be adjusted by mounting a different size pair of electrodes on the sensor).

In embodiments, the electrode pairs are removable from the sensors. In such embodiments, the plurality of sensors are configured to receive pairs of electrodes. In such embodiments, electrode pairs with a desired size and/or IED may be mounted in the sensors as required (and moved to a position as desired, for sensors where the position of the electrodes are moveable).

Accordingly, in an embodiment, the surface electromyography system of the technology described herein comprises a kit comprising:
a plurality of sensors for detecting muscular electrical activity, each sensor of the plurality of sensors operable to receive a pair of electrodes, and each sensor of the plurality of sensors operable to provide an output representing the potential difference between a pair of electrodes when mounted on the sensor; and
an output unit coupleable to the plurality of sensors, the output unit operable to provide outputs representing the outputs of each of the plurality of sensors; and
a plurality of electrodes configured for mounting within the sensors of the plurality of sensors, optionally wherein the plurality of electrodes comprises plural different sizes of electrode.

Similarly, in an embodiment, the method of measuring electrical activity over a region of the surface of a subject's skin using the system of the technology described herein comprises:
mounting a pair of electrodes within each sensor of the plurality of sensors;
attaching the plurality of sensors to the subject's skin such that the pairs of electrodes are in contact with the subject's skin;
detecting a potential difference between the pairs of electrodes; and
providing an output representative of the potential difference detected by each of the pairs of electrodes.

The electrode pairs may be provided on (as part of), for example, electrode pads which are mountable within (and removable from) a sensor housing. Each electrode pair may be provided on a (single) electrode pad. Alternatively, each electrode may be provided on a respective electrode pad (such that each electrode pair is provided as a pair of electrode pads). The electrode pads in an embodiment further comprise one or more connectors for forming a connection to the sensors. The connector(s) may (each) comprise an electrical contact (e.g. such as a snap connector), a conductive adhesive or any other suitable connector. The electrode pads may be disposable single use pads. In an embodiment, the electrode pairs of the sensors are separated by the same IED (when in use). In other embodiments, some or all of the plurality of sensors have different IEDs. For example, the system may include four sensors, two of the sensors having an IED of 15 mm and two of the sensors having an IED of 21 mm. In a second example, all of the sensors may have a personalised (and therefore potentially different) IED based on factors such as the muscle depth at their particular region. It will be appreciated that the optimum IED for each of the sensors may vary between patients, and may even vary between tests for the same patient. Alternatively, the sensors may all have the same IED.

In an embodiment, the system automatically records the parameters used for each test. For example, the system may record the IED separation of (or electrode pair selection for) each of the sensors. Alternatively, the system may comprise a means for manually recording the test parameters, such as the IED separation.

In an embodiment, the electrodes of the electrode pairs have a largest dimension between 1 mm and 20 mm, 1 mm and 15 mm, 1 mm and 10 mm or 1 mm and 5 mm. For example, the electrodes may be circular electrodes with a diameter between 1 mm and 20 mm, in an embodiment between 1 mm and 15 mm or square electrodes with side lengths within this range. In an embodiment, a circular electrode with a diameter of 18 mm is used. The IED is in an embodiment large enough that there is a gap of at least 0.5 mm or 1 mm between the electrodes of the electrode pairs. In embodiments, there is a gap between the electrodes of at least 3 mm, or at least 4 mm, or at least 5 mm. In some embodiments, the ratio between the IED and the largest dimension of the electrodes is in an embodiment between 2:1 and 5:1, for example 3:1 or 4:1. The Applicant has recognised that the spectral properties of the sEMG signal are influenced by the size of the electrodes. For example, as the diameter of a circular electrode increases, higher frequency components of the sEMG signal are attenuated. This is thought to be due to the conductive area of the electrode averaging the voltage distribution under it, and generating a smoothed version of the actual spatial potential distribution. Consequently, the electrode creates a low-pass filter whose cut-off frequency decreases with an increased surface area. However, a smaller surface comes at the expense of increased skin surface impedance, and thus, increased noise. Using an IED of each of the electrode pairs within any of the ranges discussed above, as well as in an embodiment a ratio between the IED and the largest dimension of the electrodes of between 2:1 and 5:1 has been found to provide a good balance of these various considerations.

The electrodes are in an embodiment Ag/AgCl electrodes. Ag/AgCl electrodes beneficially provide an output with a relatively low level of noise due to a low electrode-skin impedance. However, any other suitable electrode material may be used. In embodiments where the electrode pairs are be provided on (as part of) electrode pads, each electrode may comprise (may be) a layer (e.g. an Ag/AgCl layer) within an electrode pad. The sensors are in an embodiment suitable for use in a surface electromyography system, i.e. for detecting electrical potential on the surface of a subject's skin. Therefore, each sensor in an embodiment comprises a means for securing the sensor to the skin of the patient, for example an adhesive pad or layer.

In embodiments where the pairs of electrodes are provided on electrode pads (e.g. as an Ag/AgCl conductive layer of an electrode pad), each electrode pad may comprise an adhesive layer and/or a conductive gel layer, which contacts the skin of a subject in use and assists with adhering the sensor to the skin of the subject.

In embodiments, the sensors are adhered to the skin of a subject (only) by means of an conductive gel layer or an adhesive layer applied to and/or around the electrodes (e.g. as part of an electrode pad) (so that no further adhesive is needed to connect the sensor body to the subject). Alternatively, any other suitable and desired means may be provided for securing the sensor to the skin of the patient, such as an adhesive pad associated with the sensor.

When secured to the subject, the electrode pair of the sensors is in contact with the skin of the patient.

The sensors in an embodiment comprise a conductive substance to reduce the impedance of the electrode-skin contact (at least when in use). For example, a conductive gel may be applied to and/or around the electrodes (e.g. as a conductive gel layer of an electrode pad). Alternatively, or additionally an adhesive applied to and/or around the electrodes (e.g. as an adhesive layer of an electrode pad) may be a conductive adhesive. In an embodiment, each sensor comprises an electrode pad comprising an adhesive pad/layer and a pair of electrodes, e.g. protruding from a surface the pad/layer.

The sensors are coupleable to (connected to) the output unit. This connection is in an embodiment a wired connection, i.e. via a connecting wire, but may otherwise be a wireless connection such as Bluetooth. In an embodiment, each sensor is independently coupleable (connected) to the output unit, e.g. by its corresponding wired or wireless connection.

The sensors (each) in an embodiment comprise a means for connecting the sensor to the output unit, for example a wireless chip or plug for connecting a wire (e.g. such as a wire-to-board connector for connecting the wire to a printed circuit board (PCB) of the sensor). The connection means is in an embodiment on an opposite side of the sensor to the electrodes, such that it will remain accessible during use of the sensor. A wireless sensor may additionally comprise a power source such as a battery, while a wired sensor may receive power from the output unit.

The system in an embodiment processes the sEMG signals detected by the electrodes of the sensors. For example, the system may amplify and/or filter the sEMG signal to reduce signal noise and/or reduce unwanted frequency components of the signals. Each of the processing steps may be performed by hardware or by software, or by a combination of the two. For example, the system may comprise hardware components configured to amplify and filter the sEMG signals, and may further comprise software configured to perform additional filtering steps. The processing may be performed at or between the sensors and the output unit. For example, some or all of the processing may be performed at the sensors such that the sensors output the processed sEMG signal to the output unit. In an embodiment, each sensor comprises processing circuit(s) and/or processing means (processor(s)) operable (configured) to perform processing of the sEMG signals detected by the electrodes. In an embodiment, the processing circuit(s) and/or processing means of the (each) sensor are integrated with a printed circuit board (PCB) of the sensor. Alternatively (or additionally), some or all of the processing may be performed after the sEMG signal is output from the sensors, for example by components of the output unit.

The system therefore in an embodiment comprises one or more amplifiers to amplify the sEMG signals detected by the electrode pairs of the sensors.

In an embodiment the total gain for the sEMG signals of each of the sensors is at least 1000, but this gain may be varied depending on the expected output amplitude from the sensors. The input range of a typical digitiser such as an A/D converter is around +5V. Thus, a total gain may be selected in order to amplify the expected signal strength (typically around 0.1 μV-2 mV for sEMG signals) to within this input range.

The one or more amplifiers in an embodiment comprise one or more front-end amplifiers to amplify the sEMG signals. The front-end amplifier is in an embodiment a differential amplifier with an input impedance of at least 100 MΩ.

In an embodiment, the front-end amplifier only applies a moderate amount of gain (for example, a gain of around 100). Advantageously, this smaller amount of gain helps to avoid oversaturation. To achieve a sufficient total gain, as discussed above, an additional amplifier can be added. In an embodiment, a first (front-end) amplifier amplifies the sEMG signal, while a second (additional) amplifier amplifies the output of the first amplifier after the signal has been filtered (which will be discussed below). In an example, the gain for the first amplifier may be 100 and the gain for the second amplifier may be 101, resulting in a total gain of 10100.

In an embodiment, the sEMG signals of the sensors are amplified individually. For example, the system may comprise individual first and second amplifiers for each of the sensors. Alternatively, one or more of the amplifiers may be configured to amplify the outputs of all or some of the sensors. For example, the system may comprise a single first amplifier and a single second amplifier each configured to amplify the outputs from all of the sensors, or the system may comprise individual first amplifiers for each of the sensors and a single second amplifier configured to amplify the outputs from all of the first amplifiers.

The detected sEMG signal is in an embodiment processed to remove unwanted frequencies and noise. This is particularly beneficial for when examining muscles that typically produce smaller sEMG signals, as the system noise may be within the same order of magnitude of the sEMG signals. For example, in one embodiment of the technology described herein, the measurement of the abdominal muscles typically produce an average rectified signal of about 5 μV to about 10 μV during deep breathing exercises, while the noise for these measurements may be between about 1 μV and about 3 μV.

In an embodiment therefore, the system comprises one or more filters configured to attenuate unwanted frequencies in the sEMG signals detected by the electrode pairs of the sensors. Again, the sEMG signals of the sensors are in an embodiment filtered individually. For example, the system may comprise individual low and high pass frequency filters for each of the sensors. The one or more filters may instead or additionally comprise one or more filters configured to filter the outputs of all or some of the sensors. For example, the system may comprise one or more notch filters configured to filter the outputs signals from all of the sensors.

In an embodiment, the one or more filters comprises a low pass filter with a cut off frequency based on, and in an embodiment at, the frequency of highest relevant muscle harmonic. In an embodiment, the low pass filter has a cut off frequency within the range of about 400 Hz and about 450 Hz.

In an embodiment, the one or more filters comprises a high pass filter with a cut-off frequency between about 10 Hz and about 20 Hz. For example, the cut-off frequency of the high pass filter may be 16 Hz. Noise resulting from, for example, motion artefacts and electrode sliding is typically in a relatively low frequency range. The high pass filter therefore in an embodiment has removes or reduces the lower frequency signals in order to reduce the influence of these sources of noise.

Both the low and high-pass filters are in an embodiment second order filters or more.

In an embodiment, the one or more filters comprises one or more notch filters. At least one of the notch filters may be centred around the power line frequency (typically 50 Hz or 60 Hz depending on the region) in order to reduce the power line interference (PLI) on the detected signal. The system may comprise a notch filter centred at the power line frequency with additional notch filters centred at the harmonics of the power line frequency. For example, the system in an embodiment comprise notch filters centred at 50 Hz, 100 Hz, 150 Hz, 200 Hz, 250 Hz, 300 Hz and 350 Hz. It will be appreciated that different frequencies will be applicable in regions with a power line frequency other than 50 Hz.

Other techniques may be used to reduce PLI in place of or in addition to notch filters, such as spectral interpolation, and driven-right-leg (DRL) circuits.

In an embodiment, the system comprises a DRL circuit for inverting the common mode voltage of the system. A reference electrode is in an embodiment used to feedback the inverted common mode voltage to the plurality of sensors, for example by attaching the reference electrode to the skin of the subject. In an embodiment, a single reference electrode provides feedback for all of the sensors, and may be attached to the subject away from the region being tested. For example, if the sensors are detecting electrical activity of abdominal muscles, the reference electrode may be connected to the subject's arm. Beneficially, by inverting the common mode voltage of the system, the DRL circuit also inverts any common mode voltage injected by hardware components such as amplifiers or filters. For example, in systems that include front-end amplifiers, the DRL circuit also reduces interference from the common mode voltage originating from the amplifiers.

In an embodiment, the system further comprises an isolation device to isolate the sensors from devices powered by the power grid (such as the output unit), and thereby protect the circuit, user and subject.

In an embodiment, the sEMG signals are further processed to reduce artefacts and interference from, for example, electrocardiogram (ECG) signals, which will typically be around a few μV. This is in an embodiment done using wavelet filtering techniques.

The wavelets filtering in an embodiment utilises daubechies 4 (DB4) wavelets, and comprises applying threshold detail coefficients to levels 4, 5, 6 and 7 of the wavelet decomposition. The threshold is in an embodiment determined using the maximum amplitude of a relaxed signal from the muscles, as a relaxed signal contains ECG artefacts without interference from muscles. The relaxed signal is decomposed to determine individual thresholds for each of the levels 4, 5, 6 and 7. In an embodiment, the threshold is set to 10% above the maximum amplitude of the reference decomposition and all detail coefficients, which are below its specific level's threshold, are zeroed. This way, the ECG artefacts are reduced while larger signal components from e.g. muscle activity in the same frequency band are largely unaffected.

To further reduce noise in the sEMG signal, the detail coefficients in level 1 and 2 of the wavelet decomposition are in an embodiment zeroed. These detail coefficients typically contain high-frequency bandwidth components around 450 Hz and higher. The wavelet filtering therefore also beneficially reduces any remaining undesired high frequency signals above the low pass filter cut off frequency.

As discussed above, each of the processing steps may be performed via hardware components or via software. For example, each of the one or more filters may be a hardware filter or a software filter. In an embodiment, the system comprises both analogue hardware and digital software filters with the same or about the same cut-off frequencies to improve signal quality and reduce noise.

In an embodiment, the system comprises both hardware and software processing steps. For example, the processing steps may include a series of hardware processing steps prior to digitisation of the signal and a series of software processing steps post digitisation of the signal. The hardware steps may comprise any one or more of a first amplification stage, low pass filtering, high pass filtering, a second amplification stage and/or the use of a DRL circuit to invert a common mode voltage. The software processing steps may include any one or more of low pass filters, high pass filters, notch filters and/or wavelet filtering.

In an embodiment, one or more sensors of the plurality of sensors (and in an embodiment each sensor) is configured to perform analogue (e.g. hardware) processing of sEMG signals from its associated electrode pair, the analogue processing comprising one or more of (in an embodiment plural of, in an embodiment all of) a first amplification stage, low pass filtering, high pass filtering, a second amplification stage, and/or use of a DRL circuit to invert a common mode voltage (e.g. such as described above). In an embodiment, the one or more sensors (in an embodiment each sensor) is configured to (also) perform analogue-to-digital conversion (ADC) (digitisation) of the (processed) sEMG signals to generate corresponding digital (digitised) signals, and is in an embodiment configured to transmit the digital signals to the output unit. In such embodiments, the output unit may be configured to receive the digital signals, and in an embodiment perform further (e.g. software) processing of the digital signals, such as applying one or more (in an embodiment plural of, in an embodiment all of) of low pass filters, high pass filters, notch filters and/or wavelet filtering, as discussed above.

As noted above, the sEMG signals arising from abdominal muscle activity which are to be measured for the purposes of detecting peritonitis may have a small amplitude of the order of μV, and so the Applicant has recognised that it is important to mitigate against noise and other artefacts that may affect the sEMG signals. The Applicant has recognised in this regard that providing analogue signal processing close to the patient, as part of the sensors, can help to avoid introducing noise and artefacts into the sEMG signals, since the analogue signals are only handled within the sensor itself (and not transmitted over any significant distance such as to an external processor).

Furthermore, by providing analogue-to-digital conversion as part of (within) the sensors, digital signals can be transmitted to the output unit via a suitable connection, e.g. such as WiFi, Bluetooth, or a wired connection. In an embodiment, the connection is a wired connection between each respective sensor and the output unit, through which the sensors also receive power. In an embodiment the wired connection (wire) is not shielded. In this regard, the Applicant has recognised that transmission of digital signals allows a relatively thin and flexible unshielded wire to be used (compared to transmission of analogue signals which may require a thicker, shielded, wire to avoid introduction of noise and artefacts), making the sensor system relatively less bulky and easier to use. In an embodiment, the system further comprises a digitiser for converting the voltage detected by each of the sensors into a digital signal. The potential detected by the electrodes of the sensors will typically be an analogue signal, and therefore may beneficially be converted into a digital signal to assist with e.g. processing by software. The digitiser may be any suitable digitiser that is operable to digitise (convert) analogue signals received from the plurality of sensors into a digital signal, such as an A/D converter. As discussed above, each sensor may comprise a digitiser for digitising sEMG signals from its electrode pair. As discussed above, the input range of a typical digitiser is around +5V. Thus, a total gain from any amplification stages in the system (e.g. in the sensors) may be selected in order to amplify the expected signal strength (typically around 0.1 μV-2 mV for sEMG signals) to within this input range.

In an embodiment, the system has a sampling frequency of at least 1000 Hz, and in an embodiment, at or about 2048 Hz. To avoid aliasing, and according to the Nyquist sampling theorem, the minimum sampling frequency of the digitiser must be greater than twice the maximum frequency of the signal. A digitiser sampling frequency of 1000 Hz is therefore generally sufficient for most sEMG applications.

The system comprises an output unit coupleable to (connected to) the plurality of sensors. The output unit is operable to receive the detected signals from the sensors and provide an output indicative of the detected sEMG signals. For example, the output may be the processed signals detected by the sensors. Alternatively, the output may be numbers representing an average or maximum amplitude of the sEMG signal, or any other suitable method of providing an indication of the amplitude of the sEMG signals.

The output unit may provide an output that superimposes or combines the signals received from each of the sensors, but in an embodiment provides outputs for each of the sensors that may be displayed separately, for example side by side. In an embodiment, the output unit outputs results indicative of the potential detected by each of the sensors simultaneously, such that the detected sEMG signals may be directly compared to one another during a test. This comparison may be provided by software (e.g. mathematical analysis) or by the user (e.g. a visual comparison).

In an embodiment, the signals from the sensors are synchronised, such that sEMG signals detected at the same time by different sensors are aligned to a same (e.g. global) time. In an embodiment, a global time (global clock) is maintained (e.g. by the output unit) to which the sEMG signals from the sensors are synchronised. The Applicant has recognised that such synchronisation allows signals from different sensors to be directly compared, so that muscle responses (e.g. such as abdominal guarding in response to a trigger such as a cough or applied pressure) can be compared across different sensors at different abdominal positions on a patient. The Applicant has recognised that such synchronisation may be warranted particularly in arrangements where each sensor has its own circuitry for performing signal processing (thus forming a distributed processing system).

Thus, in embodiments where the sensors are configured to perform sEMG signal processing (as discussed above), each sensor in an embodiment has a local time (local clock). In such embodiments, the local time (local clocks) of the sensors are in an embodiment synchronised to a global time (global clock), in an embodiment by periodically sending a synchronisation signal to each (all) of the sensors (e.g. from the output unit). In an embodiment, during each sEMG measurement, each sensor runs off (uses) its local clock, with the local clock being updated (in response to a synchronisation signal) between sEMG measurements. In an embodiment, the local clocks of the sensors are updated between different measurements on a same patient and/or between measurements on different patients. In other words, each sensor runs off its local clock during an sEMG signal measurement (whilst obtaining sEMG signals).

The output unit in an embodiment comprises a display for displaying the output results. Alternatively, the output unit may transmit the output results to an external display unit. The external display unit may be any suitable display, for example a monitor, television, mobile device or a dedicated display system.

In an embodiment, the software for e.g. processing the sEMG signals is provided by the output unit.

In an embodiment, the output unit comprises a means for controlling the sensors and/or the processing steps. For example, the output unit may comprise a touch screen, buttons, dials or any other suitable control means. In this embodiment, the output or control unit may assist the user in activating/deactivating selected sensors, configuring the processing of the sEMG signal and/or configuring the nature of the output results.

Some diagnostic methods include evaluating a muscular response to an applied pressure (or other stimulation, e.g. such as may be provoked by a cough or other provoking means). For example, patients with peritonitis may have an involuntary muscle response in the abdominal wall (abdominal guarding) in response to provoking the abdominal wall. Whilst abdominal guarding can sometimes be felt by a doctor placing their hand on a patient's abdomen, the muscle movement is very small and can be hard to feel, requiring a highly experienced doctor.

In an embodiment, therefore, the system is configured to detect a stimulation of (a stimulation applied to, in an embodiment a force (pressure) applied to) a subject during use of the system. In an embodiment, the system is configured to provide an indication of (an output representative of) the amplitude (magnitude) and/or timing of the (applied) stimulation (e.g. force). In embodiments, the system is configured to indicate (detect) at least one of (in an embodiment plural of, in an embodiment all of): a time at which stimulation (e.g. force (pressure)) was applied to a subject (commenced) during use of the system; a time at which stimulation (e.g. force (pressure)) was released (removed) from a subject (ceased) during use of the system; and an amount (a magnitude) of stimulation (e.g. force (pressure)) applied to a subject during use of the system.

In an embodiment, the system comprises one or more force sensors (pressure sensors) to measure a force (pressure) being applied to a subject. In an embodiment, the one or more force sensors are coupleable to one or more of the plurality of sensors, and may therefore measure a pressure applied to the sensor. In an embodiment, one or more of (in an embodiment plural of, in an embodiment each of) the sensors is associated with (comprises) a force sensor. In an embodiment, the force sensors are integrated into at least one of (and in an embodiment plural of, and in an embodiment all of) the sensors, and measure a pressure applied to the (respective) sensor (and therefore, to the subject via the sensor). Thus, in an embodiment, each sensor of the plurality of sensors has its own respective (integrated) force sensor.

Any suitable and desired force sensor may be used. In embodiments, the (each) force sensor comprises a load cell. Alternatively, the one or more force sensors may comprise any suitable and desired piezoresistive or piezoelectric force sensor(s).

In this regard, the Applicant has recognised that providing a (each) sensor with a force sensor has the advantage that the location, timing, and amplitude of an applied force can be more reliably identified (e.g. compared to applying a force to an arbitrary, unknown, location of a patient). Furthermore, providing plural sensors with a respective force sensor allows force to be applied at multiple locations on a patient (e.g. in turn) without needing to move the sensors. This can allow muscle response at different locations to be compared.

Hence, in an aspect of the technology described herein, there is provided a plurality of sensors for use in a surface electromyography system for detecting muscular electrical activity, each sensor of the plurality of sensors adapted to receive a respective pair of electrodes and configured to receive signals from its respective pair of electrodes when mounted in the sensor;
each sensor of the plurality of sensors comprising a respective force detector, each sensor configured to receive force signals from its respective force detector simultaneously with receiving signals from its pair of electrodes;
each sensor of the plurality of sensors configured to output signals representative of the signals received from its respective pair of electrodes and force sensor.

In embodiments, the force sensor comprises a force sensitive button on a top side (when in use) of the sensor unit. In an embodiment, the force sensitive button comprises a button engageable with a force detector. In an embodiment, the force detector comprises a load cell. The Applicant has recognised that such a force sensor arrangement can provide a robust and reliable arrangement, which responds relatively quickly to pressure application and removal. Alternatively, other types of force sensor could be used, e.g. such as other piezoresistive or piezoelectric force sensors.

In embodiments (such as embodiments where a force sensor is integrated into a sensor of the system), the sensor (sensor unit) is configured to resist deformation in response to the applied force (aside from movement of the button in response to the applied force). In this regard, the Applicant has recognised that deformation or movement of internal components of the sensor could negatively affect the sEMG signal quality. In embodiments, the sensor (sensor unit) is rigid, in an embodiment comprising a rigid enclosure (in which the force detector and other processing circuits of the sensor are housed). In an embodiment the sensor unit enclosure is constructed of a rigid, non-conducting material such as plastic.

In this regard, by providing a non-conductive enclosure for each sensor, electrical interference from a user touching the sensor to apply force may be reduced. The sensor (or components thereof, such as the PCB) could also be electrically shielded, if desired.

In embodiments (such as embodiments where a force sensor is integrated into a sensor of the system), the sensor (sensor unit) is configured (constructed) to mitigate against the effect of pressure on the sEMG signals obtained by the electrodes. In this regard, the Applicant has recognised that deformation of the electrodes (e.g. due to pressure application) could impair sEMG signal quality. Accordingly, in an embodiment, the sensor (sensor unit, e.g. a housing of a sensor unit) comprises one or more recesses adapted to receive at least part of the pair of electrodes (electrode pad(s)), in an embodiment such that (the skin-contacting surface of) the electrodes (electrode pad(s)) lies substantially flat. This may help to reduce deforming forces on the electrodes when pressure is applied to the sensor (sensor unit). In an embodiment the one or more recesses are adapted to contain at least a part of the electrodes (electrode pad(s)) which forms an electrical connection with the sensor (sensor unit), for example such as a protruding or mating electrical connector.

In embodiments, a single recess is provided in the sensor (sensor unit) for receiving the pair of electrodes (e.g. as a single electrode pad or a pair of electrode pads). Alternatively, a pair of recesses could be provided (for receiving respective electrodes (electrode pads)), or other arrangements of recesses could be provided.

In embodiments, the one or more recesses of a (each) sensor are configured (sized) such that the pair of electrodes fit (transversely) within the one or more recesses. In embodiments, the pair of electrodes are substantially completely contained within the one or more recesses, such that a skin contacting surface of the electrodes lies substantially flush with (does not project from) the sensor unit. Accordingly, in embodiments, the one or more recesses of the sensor (sensor unit) substantially shield the electrodes from compressive forces applied to the sensor (sensor unit). In such embodiments, electrode-skin contact may be obtained when the sensor unit is positioned on a subject's skin without the electrodes being compressed.

In embodiments where electrode pad(s) are used, the electrode pad(s) and one or more recesses may be dimensioned (sized) such that the electrode pad(s) extend (transversely) beyond the one or more recesses, and are supported on (lie against) a bottom surface of the sensor (sensor unit) (whereas the electrodes in an embodiment, being smaller than the overall electrode pad size, fit within the one or more recesses). In an embodiment, the electrode pad(s) do not extend (transversely) beyond the bottom surface of the sensor (do not extend beyond the footprint of the sensor). This may assist with applying pressure evenly across the electrode pad(s). Alternatively, the electrode pad(s) could extend (transversely) beyond the bottom surface of the sensor.

Thus, the provision of one or more recesses and may help prevent pressure forces on the electrodes (electrode pads) which could affect the quality of the sEMG signal obtained by the electrodes. In embodiments where the pair of electrodes (electrode pad(s)) contain (or are provided in combination with) a conductive gel, the one or more recesses may also help prevent uneven pressure on the conductive gel, which may otherwise cause gel movement or leakage which could affect sEMG signal quality. In an embodiment, the one or more recesses are configured such that the conductive gel (and the electrodes) are (both) fit transversely within the one or more recesses, in an embodiment such that the conductive gel (and the electrodes) are (both) substantially completely contained within the one or more recesses. To (further) reduce the chance (extent) of gel movement or leakage, a conductive gel of relatively higher viscosity could be used, and in embodiments this is done.

In an alternative embodiment, a flat electrical connection may be provided between the sensor (sensor unit) and the pair of electrodes (electrode pad(s)), in which case the one or more recesses could be omitted (with the bottom surface of the sensor (sensor unit) which receives the pair of electrodes being substantially flat).

In an embodiment, a feedback system is provided (as part of the force or pressure sensor) to indicate when a suitable level of pressure is being applied. For example, the feedback system (force sensor) may monitor the applied pressure and compare the applied pressure to a threshold pressure. When the applied pressure exceeds the threshold, the feedback system (force sensor) may provide feedback, for example providing one or more (or all of) a visual feedback, an audio feedback and a haptic feedback. In an embodiment, a visual feedback such as one or more lights is provided to indicate that the correct pressure is being applied. The visual feedback (e.g. one or more lights) may be provided by a sensor unit which the force sensor is part of. Alternatively, the visual feedback may be provided by the output unit, such as on a display of the output unit. Alternatively, the feedback system (force sensor) may compare the applied pressure to a desired pressure range, and provide different indications depending on whether the applied pressure is below, above or within this range.

The results output by the output unit in an embodiment provide an indication of the time at which the pressure was applied and/or released. For example, if the output of the output unit comprises the processed sEMG signal (for example, with respect to time), the indication may comprise a dashed line at the time at which the pressure was applied and/or released. In another example, the signal line may change colour based on the detected force, or the output may additionally comprise a graph of the applied force with respect to time (wherein the graph of applied force may be displayed separately to one or more graphs for the sEMG signals, or superimposed on a graph for an sEMG signal).

The system of the present embodiments is suitable for measuring potentials as low as 1 μV. The potential measured by the electrode pairs is a summation of action potentials generated during muscle contraction. The amplitude of a sEMG signal is typically the range of 0.1 μV-2 mV, and is dependent on both the muscle and the contraction strength. For example, in one embodiment of the technology described herein the average rectified values of relaxed oblique muscle signals are generally in the range of 0.5 μV to 2 μV, whilst tense signals are in the ranges of 25 μV to 175 μV. Therefore, it can be beneficial to provide a system with a sensitivity as low as 0.1 μV. However, measurement noise is typically expected to fall within the range of 1-3 μV. As a result, in an embodiment the system has a sensitivity of, for example, 1 μV, 3μV, 5V or 10 μV, to assist in distinguishing detected signals from noise.

The electromyography system is in an embodiment a surface electromyography system suitable for use in assisting in the diagnosis of muscular conditions. For example, the system may be used to assist in the detection of involuntary abdominal guarding, which may provide an indication that a subject is suffering from e.g. peritonitis. The system may also assist in detecting any other condition that is detectable using electromyography.

A method of detecting muscular electrical activity using the system of the technology described herein in an embodiment comprises attaching the plurality of sensors to the skin of the subject, in an embodiment between the innervation zone and the muscle tendon to reduce cross-talk and other signal distortions. If the system comprises a reference electrode, this is also in an embodiment attached. The sensors are in an embodiment placed such that the electrodes are separated parallel to the muscle fibre direction.

Attaching the sensors to the subject may comprise applying a conductive gel to the subject or sensor to improve the electrode-skin contact, and/or applying an adhesive to secure the sensor in position and reduce unwanted sensor movement.

The electrode pairs of the sensors then detect muscular electrical activity across the skin of the subject. The detected signals are in an embodiment processed as described above. In an embodiment, the processing comprises amplifying the sEMG signal with a first amplifier, applying a low pass filter to attenuate signals above a first cut-off frequency, for example about 450 Hz, applying a high pass filter to attenuate signals below a second cut-off frequency, for example 16 Hz, amplifying the signals with a second amplifier such that the total gain of the first and second amplifiers is at least 1000 and then digitising the sEMG signal. After digitisation, the signal is in an embodiment further processed by software. The software processing in an embodiment comprises applying second low and high pass filters with the same first and second cut-off frequencies, applying a notch filter to the signal centred at the power line frequency, for example 50 Hz, to reduce PLI, and finally applying wavelet filtering techniques. The output unit then outputs the processed signals, and in an embodiment provides a separate output for each of the plurality of sensors.

The method in an embodiment comprises a series of tests. The tests may include, for example, detecting an electrical potential of relaxed muscles, detecting an electrical potential of tensed muscles, detecting residual electrical potential after relaxation of tensed muscles, detecting an electrical potential of the muscles during and after involuntary movement (stimulation) (such as a cough), detecting an electrical potential of the muscles during conscious deep breathing by the subject, and/or detecting an electrical potential of the muscles after applying pressure to a part of the subject's body.

In an embodiment, applying a pressure to a part of the subject's body comprises applying a pressure with, or via, one or more force sensors suitable for detecting the applied pressure. The force sensor may be attached directly to the subject's body or may otherwise be attached to the subject's body indirectly, for example by connecting the force sensor to one or more of the plurality of sensors.

The detected signals may provide several means for assisting in the detection of involuntary abdominal guarding. In patients with peritonitis, for example, a higher resting activity in the abdominal muscles is to be expected, along with increased residual activity after voluntary and involuntary muscle contractions (as may be stimulated (provoked) by, for example, tensing and/or coughing and/or pressure application).

In an embodiment, a method of using the system described herein comprises determining based on the output representative of the potential difference detected by any one or more (or all) of the pairs of electrodes of the one or more of the sensors, one or more conditions indicative of a medical condition such as peritonitis.

In an embodiment, determining one or more conditions indicative of a medical condition (such as peritonitis) comprises comparing the output (from one or more, or all, of the sensors) against an expected baseline activity (for a healthy patient), and in an embodiment detecting a condition when the output deviates substantially from the expected baseline activity. In an embodiment, determining one or more conditions indicative of a medical condition (such as peritonitis) comprises, comparing the output (from one or more, or all, of the sensors) against one or more thresholds (wherein, in an embodiment, muscle activity indicated in the output(s) above a threshold level indicates a medical condition, such as peritonitis).

In an embodiment, determining one or more conditions indicative of a medical condition (such as peritonitis) comprises identifying a residual muscle activity after eliciting muscle stimulation (such as after applying a pressure to, or eliciting a cough from, a patient). In an embodiment residual muscle activity of an amplitude and/or duration greater than a threshold amplitude and/or duration is determined to indicate a medical condition, such as peritonitis.

The determining one or more conditions is, in an embodiment, done by a user visually inspecting the output data (for example, visually inspective graphs displayed by the output unit, showing output data from one or more, or all, of the sensors). Alternatively, the determining one or more conditions may be performed (at least in part) by the output unit.

The output unit, in this regard, may comprise a computer configured to perform determinations based on the output data (such as described above). The processing performed by the output unit (computer) may be performed using appropriately configured processors.

The output unit (computer) may be implemented consistent with a general purpose or special purpose computing system. Example computing systems include, but are not limited to, software or other (e.g. hardware) components within or embodied on personal computing devices, portable (e.g. hand-held) or laptop devices, medical computing devices, microprocessor systems and distributed computing systems (e.g. such as cloud based computing systems). The output unit may comprise one or more processors and one or more machine-readable memories in communication with the one or more processors. The processor(s) may assist with receiving data from the one or more sensors, performing processing of the data, and displaying the data. The memory may store instructions to cause the output unit (and processors thereof) to perform desired processing and display of the data.

A number of embodiments of the technology described herein will now be described by way of example only and with reference to the accompanying drawings, in which:

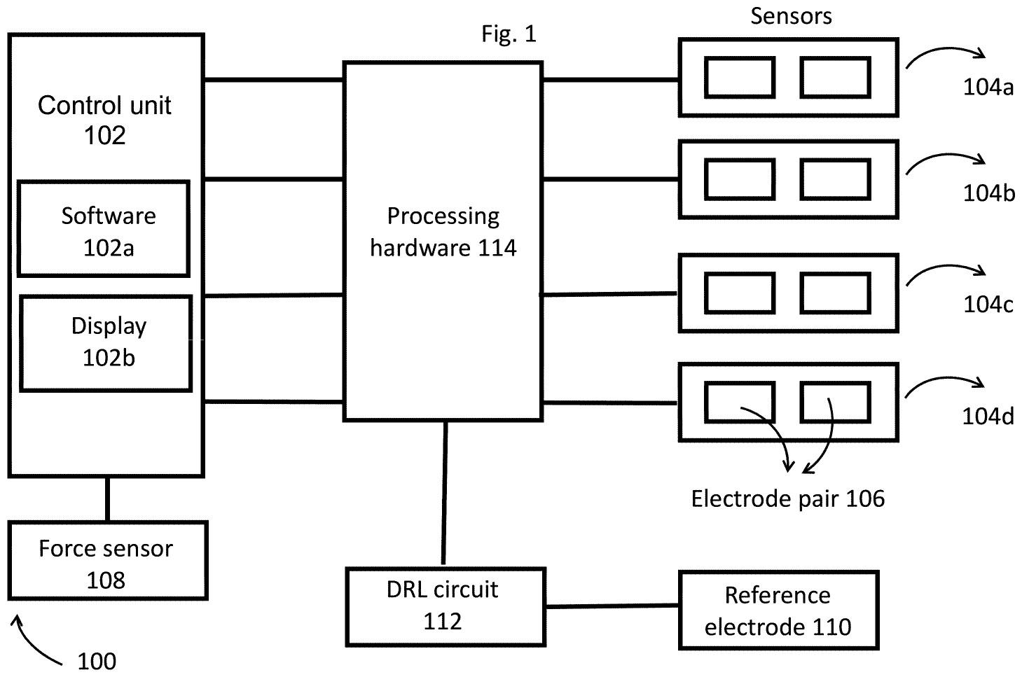

FIG. 1 is a schematic diagram of an electromyography system according to an embodiment of the technology described herein.

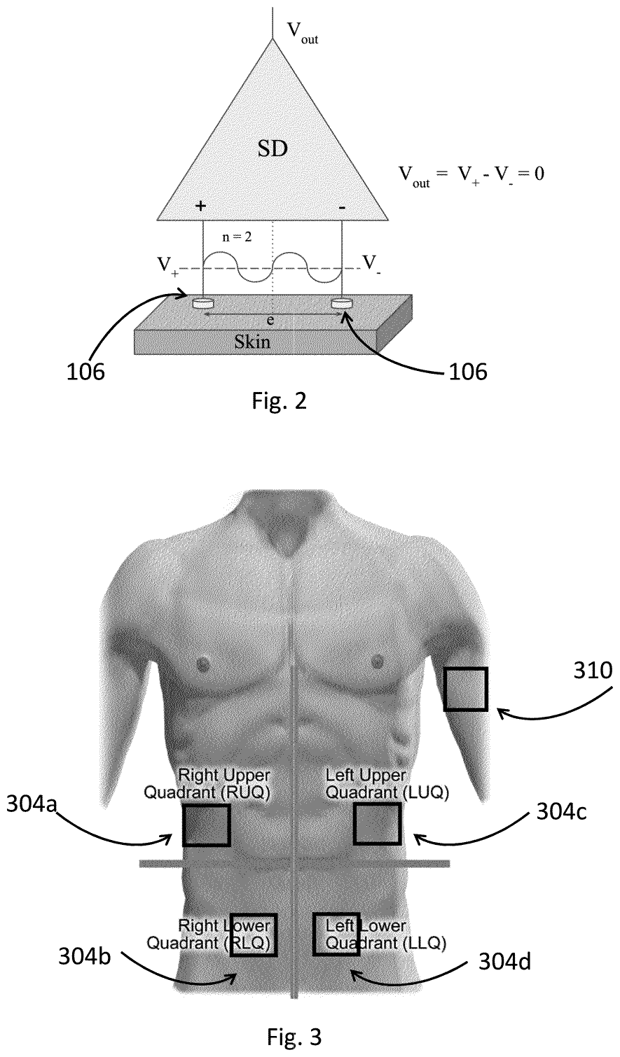

FIG. 2 is an illustration of a sensor electrode configuration according to an embodiment of the technology described herein.

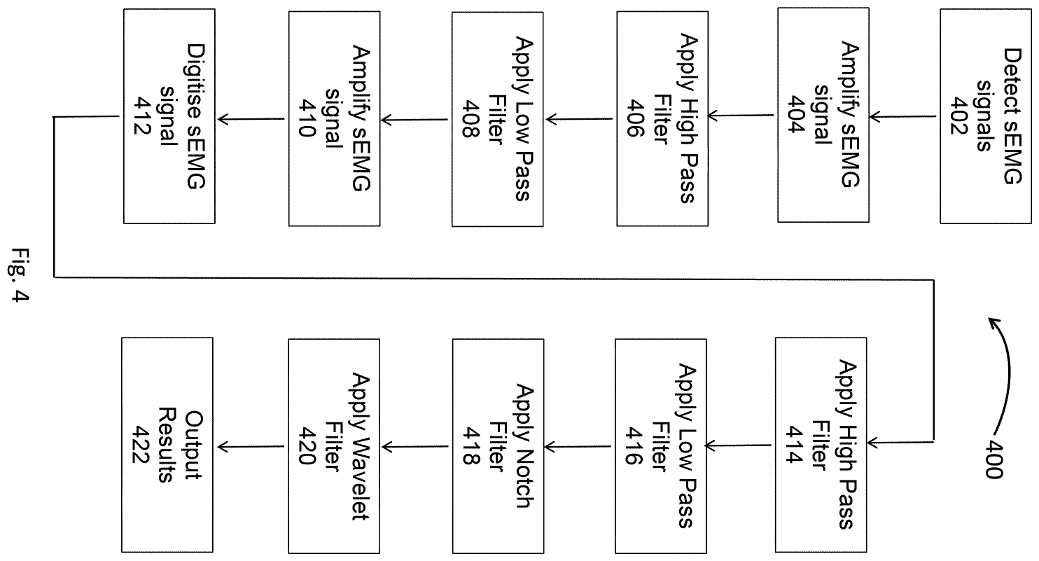

FIG. 3 is a picture showing an example of potential sensor placements during use according to an embodiment of the technology described herein.

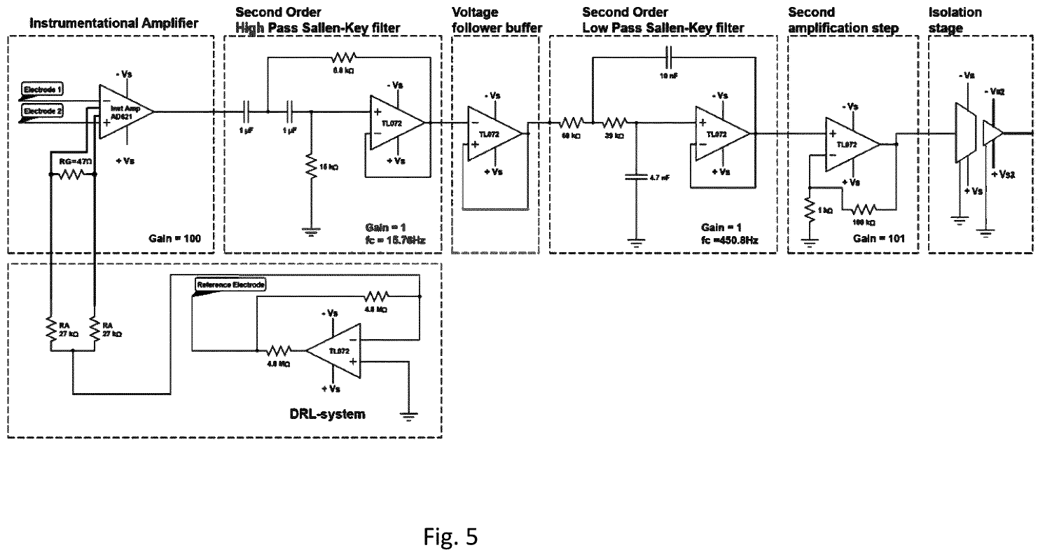

FIG. 4 is a flow chart showing example signal processing steps performed in an embodiment of the technology described herein.

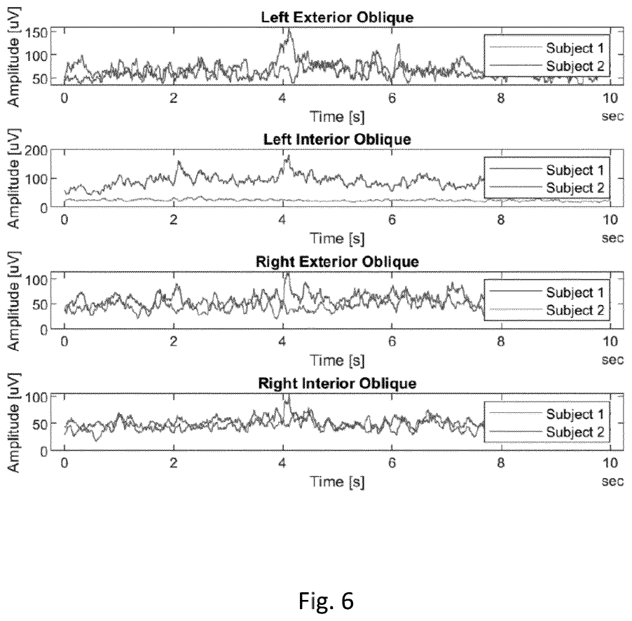

FIG. 5 is an example circuit diagram of a sEMG sensor according to an embodiment of the technology described herein.

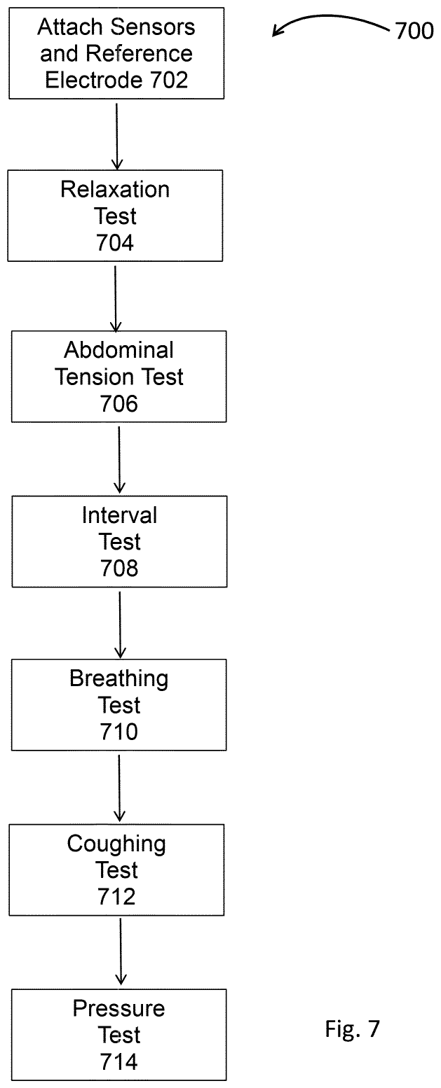

FIG. 6 is a set of output results according to an embodiment of the technology described herein.

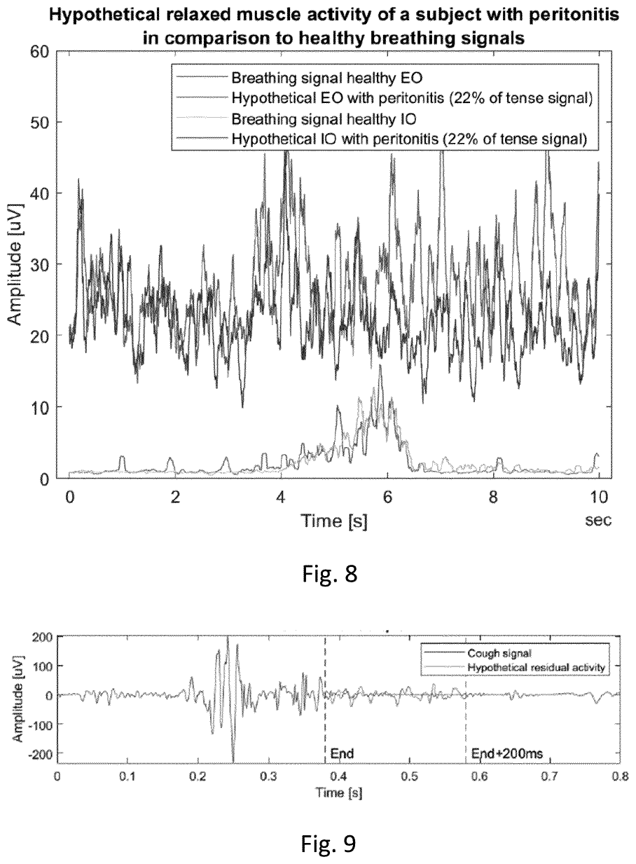

FIG. 7 is a flowchart of a method of measuring muscular electrical activity according to an embodiment of the technology described herein.

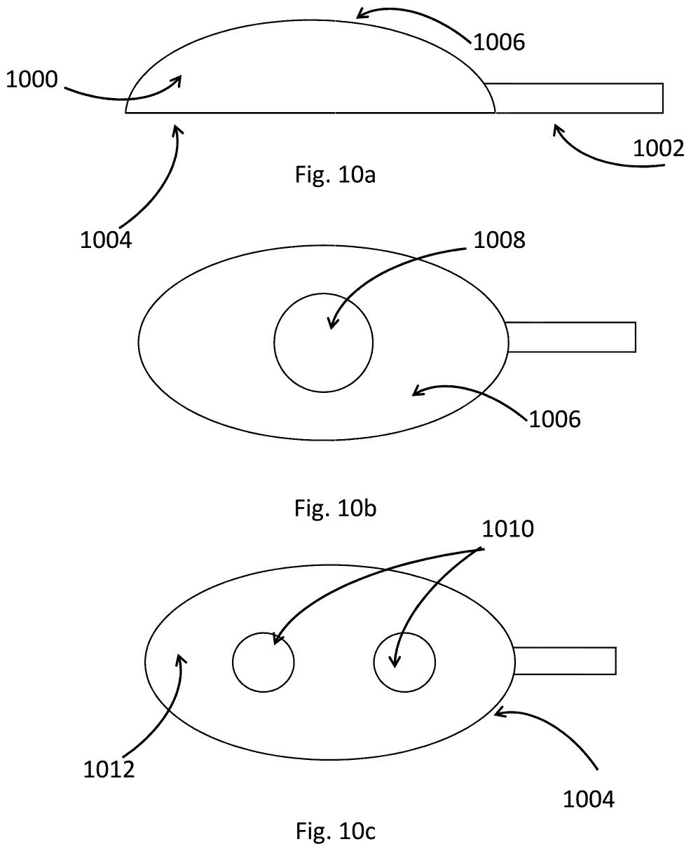

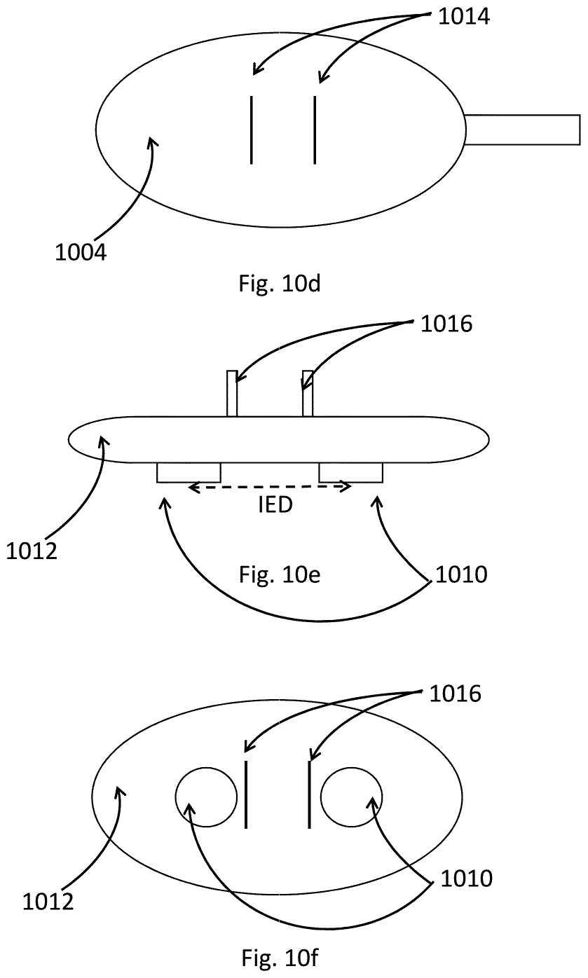

FIGS. 8 and 9 are example outputs according to an embodiment of the technology described herein.

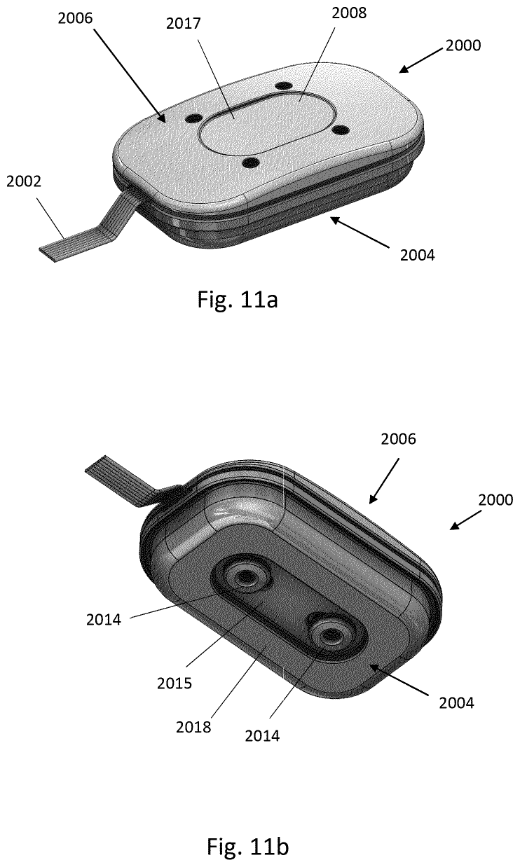

FIG. 10a-f are views of a sensor according to an embodiment of the technology described herein;

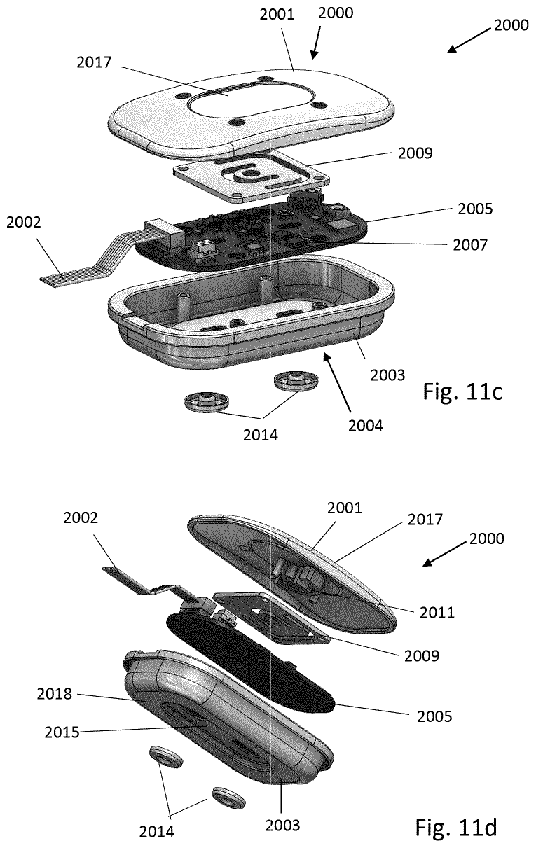

FIG. 11a-e are views of a sensor according to another embodiment of the technology described herein;

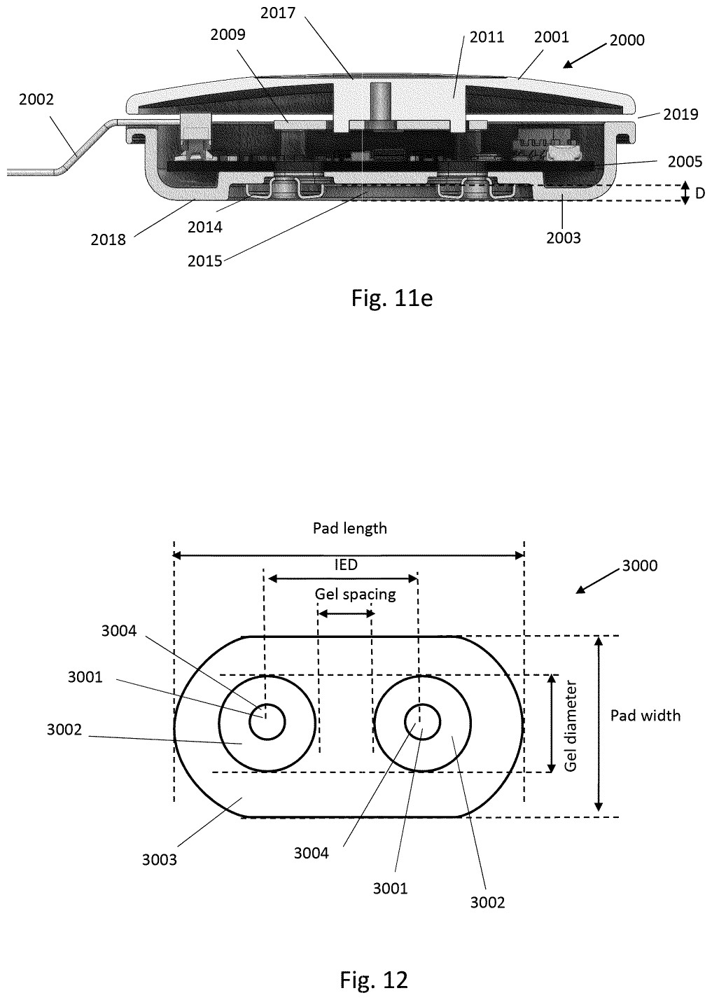

FIG. 12 is an example electrode pad as may be used in embodiments with the sensor of the technology described herein;

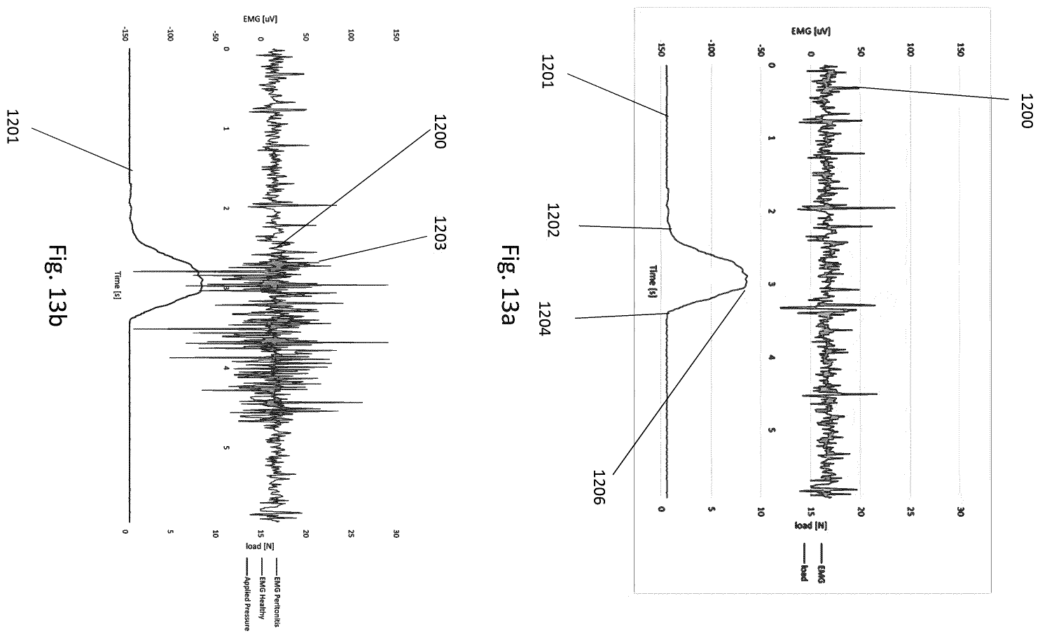

FIGS. 13a-b are example output results according to an embodiment of the technology described herein, in which an applied pressure is indicated.

Like reference numerals are used for like components where appropriate in the Figures.

An embodiment of the technology described herein will now be described.

FIG. 1 shows schematically an arrangement of an embodiment of the electromyography system according to an embodiment of the technology described herein. This embodiment of the electromyography system is primarily intended for use in measuring electrical activity of the abdominal muscles, specifically the external obliques and the internal obliques. For example, the embodiment shown in this figure may be used to detect electrical activity of the abdominal muscles during a diagnostic test for peritonitis. However, it will be understood that the same or similar electromyography system designs may be used to detect electrical activity in any other skeletal muscles, and particularly for surface muscles.

Electromyography system 100 comprises an output unit 102 connected to a set of sensors 104a-d. While electromyography system 100 is shown with four sensors, it will be appreciated that more generally the system 100 may comprise any number of sensors suitable for the intended use.

Each sensor includes a pair of electrodes 106 arranged in a single differential (SD) configuration, as shown in FIG. 2. The electrodes may be any suitable electrodes, for example, the electrodes in an embodiment have largest dimensions between 1 mm and 15 mm, and further in an embodiment are Ag/AgCl electrodes. For example, the electrodes may be circular electrodes with a diameter of 10 mm. By comparing the potentials of two adjacent electrodes 106, the sensor measures the potential difference between two points of the subject's skin. Generally, the two points should be aligned with the direction of the muscle fibres beneath the skin at those points, and on the same side with respect to the innervation zone. By taking the voltage difference between two points, the SD electrode configuration reduces interference from common mode signals. Compared to other electrode configurations such as mono-polar (MP) configurations, the SD configuration is less sensitive to noise, but can only detect superficial and near sources. The SD configuration is therefore primarily suitable for use when measuring the electrical activity of surface muscles. Other electrode configurations, including MP and double differential (DD) configurations, may also be used.

In the SD configuration, the electrodes 106 are separated by an inter-electrode distance (IED) e, as shown in FIG. 2. An IED over 10 mm can distort the electromyography signals due to a band-pass spatial filter introduced by the SD configuration. In addition, smaller IEDs reduce the cross-talk between sensors 104a-d. As a result, a smaller IED is generally preferable. However, a smaller IED also generally reduces the amplitude of the electromyography signals, as a smaller length of muscle is being examined. This can be a particular issue with regard to patients with a large amount of subcutaneous tissue (such as fat) at the sensing site, as this may increase the distance to the muscles and thereby reduce the amplitude of the surface electromyography signals.

Therefore, the IED may generally be within a range of 3 mm and 50 mm, and in an embodiment between 3 mm and 40 mm, 3 mm and 20 mm, or 3 mm and 5 mm. In one embodiment, each sensor may have a fixed IED.

Alternatively, sensors 104a-d may include electrode pairs 106 with a variable separation distance, such that a suitable IED may be selected. In this case, the IED is in an embodiment fixable at a range of IED values such that the IED can be fixed when the sensors are in use.

In a further alternative, the sensors 104 may be configured such that the pairs of electrodes 106 are removable, and electrode pairs 106 with different IEDs may be mounted on the sensor. For example, the electrode pairs 106 may be provided on electrode pads, and the sensors 104 may include a housing with a mount suitable for holding the electrode pads.

During use, each of the sensors 104 are attached to the body of the patient, for example using an adhesive pad that forms part of the sensors or any other suitable means. FIG. 3 shows an example of the sensor configuration during a test for peritonitis. Sensors 104a-d are used to simultaneously detect electrical activity in the left and right hand external and internal obliques. Each of the sensors 104a-d may be positioned in one of the abdominal quadrants shown in FIG. 3, for example in locations 304a-d, while a reference electrode 110 may be positioned away from the test area, for example at location 310.

Output unit 102 is coupled to sensors 104a-d and records the measurements taken by each of the sensors 104, and provides an output indicative of these measurements. Output unit 102 may comprise software 102a suitable for processing the sEMG signals output from sensors 104a-d. The output unit may include a display 102b for displaying the output results, or may be transmit the results to an external display unit such as a monitor, mobile device or television.

The system 100 may optionally include one or more hardware components 114 for processing the detected signals. Each of the hardware components 114 may be integrated into sensors 104a-d or output unit 102, or may be provided independently of the sensors 104 and output unit 102.

The system 100 may optionally include driven-right-leg (DRL) circuit 112. The DRL circuit 112 inverts the common mode voltage of the system and feeds the inverted common mode voltage back to electrodes 106 via the reference electrode 110, which is also connected to the subject's body as discussed above. The inverted common mode voltage can be fed back to all of the sensors 104 via a single reference electrode 110. As a result, in an embodiment the DRL circuit 112 inverts a common mode voltage based on the signals from only one of the plurality of sensors (for example, sensor 104d as shown in FIG. 1). DRL circuit 112 advantageously reduces power line interference (PLI) without attenuating the biopotential signal, thereby further reducing signal noise. Other components or techniques can be incorporated into system 100 in addition to or in place of DRL circuit 112 to reduce PLI, such as notch filters or spectral interpolation.

Optionally, system 100 may include one or more force or pressure sensors 108. Force sensors 108 may be any suitable sensor for measuring a force applied to the subject, such as a resistive sensor. In an embodiment, the sensor is operable to measure an applied force equivalent to a weight of between 0 and 100 lb (0-45.5 kg). One non-limiting example of a suitable force sensor is a Flexiforce A201 resistive force sensor. Force sensor 108 can be used independently from sensors 104 to measure the force applied to the subject, or, alternatively, force sensor may be coupleable to one or more of the sensors 104. For example, force sensors 104a-d may be mounted on sensor 104c to measure a force applied to sensor 104c.

Force sensor 108 in an embodiment provides feedback to the user, such as a light, to indicate when a desired pressure has been applied to the subject.

The surface electromyography (sEMG) signals detected by the electrode pairs may be processed via a series of processing steps. These steps may include hardware processing steps and software processing steps. Flow diagram 400 of FIG. 4 shows an embodiment of processing steps that may be applied in a typical surface electromyography test using system 100. However, it will be appreciated that these steps may generally occur in any order and/or may be skipped, while additional processing may also be performed.

In an initial step 402, the sensors 104 detect the electrical activity of the targeted muscles. The detection comprises detecting a potential difference between the electrodes of the pairs of electrodes, for example by determining a difference between the electrical signals from each of the electrodes. This detected potential forms the sEMG signal, and will vary over time.

In step 404, the sEMG signal detected by the electrode is amplified in an amplification by a front-end amplifier such as a differential amplifier. Such a differential amplifier may also be used to determine the difference between the electrical signals from each of the electrodes, as discussed in step 402. The front-end amplifier may be any suitable amplifier operable to apply a sufficient gain to the signals, such as an Analogue Devices AD621 amplifier. The signals of for a surface electromyography test are typically in the range of 0.1 μV-2 mV, and may therefore beneficially be amplified to increase the peak-to-peak amplitude such that it is in the input range of a typical digitiser. The input range of a digitiser is often around +5V, and thus a total gain of at least 1,000 is in an embodiment used. However, the preferred gain depends on the expected signal amplitude, which in turn depends on the muscles being examined. For example, abdominal muscles such as the obliques typically produce a surface electromyography signal of about 5 μV to about 10 μV during exhalation, and hence a gain of 100,000 or more may be used for some examinations.

Following the initial amplification 406 of the signals, the signals are processed to remove unwanted frequencies and noise. Noise that is due to, for example, motion artefacts and electrode sliding, is typically in a relatively low frequency range. Therefore, in step 406 the high pass filter is used to reduce, or ideally remove, lower frequency signals. The cut-off frequency of the high pass filter may be in the range 10-20 Hz. In this example, system 100 utilises a high pass filter with a cut-off frequency of approximately 16 Hz.

In the second filtering step 408, a low pass filter is used to remove signals with a frequency above approximately 450 Hz. In general, for abdominal muscles, the highest muscle harmonic of relevance is in the range of 400-450 Hz. Therefore, more generally the cut-off frequency of the low pass filter may be between 400 Hz and 500 Hz or in an embodiment between 400 Hz and 450 Hz. It will be appreciated that these values are merely illustrative, and that the frequency of the highest relevant muscles harmonic will depend on the muscles being examined. In an embodiment, both low-pass filter and high-pass filter are at least second order filters. The filters may be any suitable low and high-pass or band-pass filters operable to cut-off frequencies below 10-20 Hz and/or above 400-500 Hz. One non-limiting example of a suitable filter is Sallen-Key filter, and in particular a second order Sallen-Key filter.

Following frequency filtering steps 406 and 408, the sEMG signal may be further amplified in a second amplification step 410. The second amplifier may be any amplifier operable to apply a sufficient gain, such as a Texas Instruments TL072CP. Beneficially, including a second amplification step 410 means that a lower gain may be applied during the first amplification step 404, thereby avoiding or reducing saturation of the signal. In this example, first amplification step 404 applies a gain of 100, while the second amplification step 410 applies a gain of 101, for a total gain of 10,100 across steps 404 and 410.

It will be appreciated that while the filtering steps 406 and 408 are shown to occur between the amplification steps 404 and 410 in this example, they may instead occur before or after any amplification of the signals. Similarly, the ordering of filtering steps 406 and 408 may be reversed, with the low pass filtering step 408 occurring before the high pass filtering step 406.

In step 412, the sEMG signals are digitised using any suitable digitiser, for example an A/D converter. The detected sEMG signals are typically analogue signals. As a result, the digitisation of these assists with further processing of the signals by software, such as software 102a of output unit 102. To avoid aliasing, according to the Nyquist sampling theorem, the minimum sampling frequency must be greater than twice the maximum frequency of the signal. Therefore, generally the digitiser may have a sampling frequency of 1000 Hz or more. For example, the digitiser may have a sampling frequency of 2048 Hz.

Following digitisation, further processing steps may be applied to the digitised sEMG signal.

In steps 414 and 416, the digitised signal is filtered by digital filters to further reduce noise and unwanted artefacts. The high and low pass filters in an embodiment have approximately the same cut-off frequencies as the hardware analogue high and low pass filters. Steps 414 and 416 may be implemented by a single band-pass filter, and in an embodiment by a second order band-pass filter, or by separate high and low pass filters.

In step 418, a notch filter is applied to remove PLI. In an embodiment, step 418 includes multiple notch filters each centred around the power line frequency and its harmonics. For example, in regions with a power line frequency of 50 Hz, step 416 may include notch filters centred about 50 Hz and optionally one or more of its harmonics of 100 Hz, 150 Hz, 200 Hz, 250 Hz, 300 Hz and 350 Hz. Similarly, in regions with a power line frequency of 60 Hz, step 416 may include notch filters centred about 60 Hz and optionally one or more of its harmonics of 120 Hz, 170 Hz, 240 Hz, 300 Hz, 360 Hz and 420 Hz. It is noted that many notch filters will change the waveform of the electromyography signal, however, in the technology described herein this disadvantage is reduced as in many applications the property of interest is the amplitude of the signal, rather than the shape.

In step 420, the sEMG signal is further processed using wavelet filtering techniques to reduce artefacts and interference from, for example, electrocardiogram (ECG) signals, which will typically be around a few μV. In an example, the wavelets filtering utilises daubechies 4 (DB4) wavelets, and the processing comprises applying zeroing all detail coefficients for levels 4, 5, 6 and 7 which are above a threshold set based on the maximum amplitude of a relaxed signal from the muscles. The threshold may be, for example, around 10% above this maximum amplitude.

To further reduce noise in the signal, the detail coefficients in level 1 and 2 of the wavelet decomposition are in an embodiment zeroed. These detail coefficients typically contain high-frequency bandwidth components of 450 Hz and higher. The wavelet filtering therefore also beneficially reduces any remaining undesired high frequency signals above the low pass filter cut off frequency.

After processing, the output unit outputs results in step 422. The output unit in an embodiment provides a separate output for each of the sensors 104a-d. For example, the output may comprise four graphs showing the processed sEMG signals from each of the sensors with respect to time. Alternatively, the output may comprise, for example, a numerical value representing an average or maximum sEMG signal detected by each of the sensors 104a-d. In a further alternative, the output unit may output the raw SEMG data detected by the sensors 104a-d without any additional processing.

Each processing step 404-422 may be performed by hardware or by software. In example system 100 of FIG. 1, processing steps 404-410 (i.e. before digitisation) are performed by hardware components of system 100 while processing 414-420 (i.e. post digitisation) are performed by software 102a of output unit 102. However, any software processing may instead be performed by e.g. software provided on sensors 104a-d, and sensors 104a-d may output a processed signal suitable for display.

FIG. 5 shows an example circuit diagram showing suitable hardware components for performing steps 404-422 as described above. Each of the components may form part of the sensors 104 or the output unit 102, or may otherwise be arranged between sensors 104 and output unit 102. In an embodiment, separate components for performing each of steps 404-410 are provided for each of sensors 104a-d. A digitiser 412 is in an embodiment also provided for (within) each of sensors 104a-d. Alternatively, a single digitiser 410 may be provided to digitise the signals received from all of the sensors 104a-d. Optionally, the system 100 may further comprise an isolation stage prior to digitisation to isolate the circuit from devices powered by the power grid, and thereby protect the circuit, user and subject. The isolation stage may be any suitable device operable to isolate the circuit, such as a Texas Instruments ISO122.

In addition to the amplifiers, filters and isolators, FIG. 5 shows an example DRL circuit 112. In addition to PLI, many components such as amplifiers output a common mode voltage. Therefore, it can be beneficial to provide a DRL circuit 112 to invert the common mode voltage from the system and feed the inverted common mode voltage back to electrodes 106 via reference electrode 110. As discussed above, the inverted common mode voltage can be fed back to all of the sensors 104 via a single reference electrode. As a result, in an embodiment only one of the plurality of sensors 104 includes a DRL circuit 112.

In this example system 100, the supply voltage, Vs, is provided using two 9V batteries which results in a range of +9V. The second supply voltage, Vs2, is provided by the digitiser and supplies the isolation stage with +15V. However, other power sources such as mains electricity may instead be used.

An example of an output of system 100 is shown in FIG. 6.

FIG. 6 shows example output results of the four abdominal quadrants during tension from two subjects. In this example, the output comprises the processed data collected during a ten second examination. It will be appreciated that the data may be processed in any way familiar to the skilled person to provide a suitable output. For example, the output may comprise a numerical value representing an averaged or maximum amplitude of the signal. In an embodiment, the output comprises a separate result for each sensor 104, but may alternatively provide a single averaged result or superimpose results from each of the sensors 104 in a single graph.

By providing simultaneous results from all of the sensors, the technology described herein beneficially provides a more accurate means by which to compare the signals. For example, the signals from the sensor on the left interior oblique of subject 2 are unusually low relative to the signals from the other sensors. This may be indicative of a problem with the subject's left interior oblique, or more likely may indicate that the sensor is poorly positioned and thus providing poor results. Without the ability to compare simultaneous live signals from each of the sensors, as provided by the technology described herein, it would not be possible to identify this poor placement of the sensor, as the measured amplitude will typically vary between measurement sessions and even between individual contractions of the muscles. In addition, as no relocation of sensors is necessary, the signals can be acquired faster and with less discomfort for the subject, and without the introduction of sources of error related to the relocation of sensors, such as any movement of the subject which may result in impaired signal quality. The sensitivity of the system means that any movement from the subject can result in disturbed signals, which render the interpretation of the signals more difficult. Relocation of sensors therefore increases the risk of subject movement artefacts.

Furthermore, the use of the plurality of sensors facilitates the signal processing and later interpretation of the data as any one or more of the sensors can be used as a reference signal for the subject. The system is therefore able to compare the sensing locations, assisting in the interpretation and visualisation of the difference between normal and pathological muscle responses. This difference can be demonstrated live, in real time, in contrast to previous systems.

Moreover, the use of multiple sensors enables the optimisation of the electrode diameter and IED for each sensing location, thereby assisting with a specific evaluation at each location. As multiple sensors are used, the IED can be as short as needed to detect muscle activity in clearly defined area. In contrast, systems with a single sensor require electrodes with a greater IED. The smaller IED of the technology described herein enabled by the plurality of sensors assists in the detection of smaller changes within the sensing regions. This beneficially means that the sensors of the technology described herein have a sufficient sensitivity for a live comparison between the sensors of the system to detect changes in the electrical activity of the muscles resulting from involuntary activity. In addition, a smaller IED beneficially reduces noise from e.g. the electrical activity of adjacent muscles.

Furthermore, the use of multiple measurement points simultaneously assists in the detection of abnormalities such as outliers and signal errors.

The use of multiple sensors therefore assists the user in differentiating between voluntary and involuntary muscle activity, as well as signal noise or other abnormalities, thereby improving the detection of pathological activity due to illness.

FIG. 7 is a flow diagram 700 showing an example of a process for measuring electrical activity of abdominal muscles using the electromyography system of the technology described herein. For example, the following steps may be applied during a test to assist in the diagnosis of peritonitis. However, the present system is not limited to measuring electrical activity of abdominal muscles, and any one or more of the below steps may be applied to measure the electrical activity of any muscle, particularly surface muscles. At each stage of the method, the detected electromyography signals are processed and results are output as described above.

In step 702, the plurality of sensors and reference electrode are attached to the subject, for example using adhesive pads. To acquire accurate and stable measurements it is important that the electrode-skin impedance is low, as generally electrode-to-skin noise is the key source of noise in sEMG applications. Step 702 may therefore comprise suitable skin preparations, such as applying conductive gel between the electrode and skin to further reduce impedance and/or rubbing the skin with an abrasive paste to remove dead cells and minimize the thickness of the skin.

In step 704, measurements are taken with the muscles in a relaxed (i.e. not tensed or contracted) state. As there is no muscle activity in a healthy subject in this state, any activity is the result of remaining noise, and step 704 may therefore provide a baseline for test noise. For example, the noise may be within the range of 0.5-2 μV during this step for abdominal muscles. However, the resting activity of a patient suffering from peritonitis is approximately equal to 22% of the activity seen in a healthy subject during a voluntary contraction. Therefore, while the relaxed healthy signals are in the range of 0.5-2 μV, the range of a hypothetical peritonitis signals varies between approximately 30-70 μV. This difference is easily distinguishable with the sensitivity of the system of the present embodiments.

In step 706, measurements are taken during voluntary muscle contraction. For abdominal muscles, the detected amplitudes are typically in the range of 25-175 μV. However, it should be noted that the measured voluntary contraction strength varies between subjects, contractions, measurement sessions, and electrode positions.

In step 708, interval measurements can be made by having the subject alternate between contracting and relaxing the abdominal muscles. In a subject suffering from peritonitis, the detected signals may indicate a higher level (e.g. amplitude) of residual activity and/or a longer decay period of the amplitude of the detected activity when transitioning from the tensed to relaxed states. The decay pattern of the signal's amplitude may therefore provide a useful indication in some tests.

In step 710, measurements may be taken during a period of conscious deep breathing by the subject. In a healthy patient, deep breathing may generate activity of approximately 10 μV, which is visible over the system noise of 0.5-2 μV as discussed above. In contrast, for a patient suffering from peritonitis the breathing activity is unlikely to be visible due to the higher baseline relaxation activity of the abdominal muscles.

In step 712 measurements are recorded during a cough. The cough activity, may typically last for 200-300 ms and have an amplitude between 100-500 μV. The cough period may be identified visually from the output results. A patient with peritonitis may have residual electrical activity for 200 ms after the initial cough activity.

In step 714, a force or pressure is applied to one or more of the sensors and a corresponding signal is observed in the opposite sensor. For example, if pressure is applied to the right hand side of the subject, electrical activity associated with muscle tension may be observed in the left hand detectors upon release. The application of the pressure may be aided via the use of a force sensor 108 configured to detect when a suitable pressure has been applied. The force sensor 108 may provide the output unit with an indication of the time, duration and/or amount of pressure applied to the subject, and the output unit may include this indication in the output results. Optionally, force sensor 108 may be mounted to one or more of the sensors 104.

As discussed above, various tests 704, 706, 708, 710, 712, 714 can cause a muscle response indicative of peritonitis, since the various actions (such as relaxed breathing, abdominal tension, deep breathing, coughing, and pressure application) can each provoke the peritoneal surface and cause involuntary muscle contractions in a patient suffering with peritonitis. Whilst the process of FIG. 7 sets out plural tests 706, 708, 710, 712, 714 which could all be performed, alternatively (only) of one or plural of the tests set out in FIG. 7 could be performed, or other tests to provoke the peritoneal surface could be performed.

FIGS. 8 and 9 show examples of how the output signal may be used to assist in the diagnosis of conditions such as peritonitis. In FIG. 8, a hypothetical peritonitis signal from measurements made on the internal and external obliques (IO and EO) is shown compared to a healthy breathing signal from measurements on the same muscles. As discussed above, an sEMG signal from a healthy patient is expected to be on the same order of magnitude as the system noise (approximately 0.5-2 μV), and even during deep breathing exercises the signal from the hypothetical healthy patient rarely exceeds 10 μV. In contrast, the hypothetical sEMG signal from a patient suffering from peritonitis is expected to be in the range of 20-50 μV. As a result, sEMG signals above a threshold of, for example, 30-40 μV during a relaxation period may provide an indication of peritonitis. Similarly, extended periods above 20 μV over the relaxation period provide another indication that may be used to diagnose peritonitis.

FIG. 9, in contrast, shows a measured cough signal beginning at about 0.2 s and ending at about 0.4 s. In a healthy patient, the measured cough signal ends almost immediately, with little to no residual activity. However, in a hypothetical sEMG signal from a peritonitis patient, the sEMG signal is expected to show residual activity for approximately 200 ms after the end of the cough. Therefore, residual activity after involuntary muscle contractions (such as coughing) or voluntary muscle contractions (such as tensing) may provide further indications of peritonitis.

FIG. 13a is a graph showing an example sEMG 1200 output signal from a sensor for a healthy patient, and force signal 1201 output from a force sensor integrated within the sensor unit (to which force has been applied force during the sEMG measurement) with respect to time.

From the graph, the time at which pressure has been applied 1202 and released 1204 can be seen. The magnitude of the pressure force applied 1206 can also be seen. The force has been applied for a time period of ˜1 second, however, the force could be applied for a longer period of time if desired.

Alternatively, instead of displaying the entire force sensor output on the graph with respect to time, an indication of the time at which force is applied and/or released could be indicated. Other indications with regards to timing and magnitude of force applied could also or alternatively be provided.

The example shown in FIG. 13a is an sEMG signal measured for a healthy patient 1200, in which pressure application does not cause any residual muscle activity above and beyond the baseline muscle activity.

FIG. 13b shows similarly the sEMG signal for a healthy patient 1200, and the force applied 1201. FIG. 13b additionally shows a hypothetical sEMG signal for a patient with peritonitis 1203. As shown in FIG. 13b, in a patient with peritonitis, muscle activity may be present during pressure application, and residual muscle activity may be present after pressure application (similarly to the residual activity seen after coughing in FIG. 9).

However, in comparison to a cough test, an advantage of applying pressure is that the particular time at which pressure is applied (and released) can be determined, making it easier to interpret when abdominal muscles have been provoked (whereas it can be difficult to determine the time at which the cough has ended).

For the data shown in FIGS. 13a and 13b, pressure has been applied to the sensor which is detecting the sEMG signals by means of an integrated pressure sensor such as discussed herein. As discussed herein, the sensor may be configured to reduce disturbance (e.g. noise or other artefacts) in the sEMG signal that could arise from applying pressure to the sensor. As a result, as shown in FIG. 13a for example, such configurations may result in the pressure application having little effect on the quality of the sEMG signal. As shown in FIG. 13b, the pressure release is, in embodiments (such as those disclosed herein), (significantly) quicker than the expected duration of residual muscle activity. If the sEMG signals are affected by pressure application, then quick pressure release can help to avoid (continuing) disturbance.

It will be understood that these indications are merely examples for a single muscular condition, and that the technology described herein may instead be used to assist with the diagnosis of numerous other conditions, each of which may display different or the same sEMG signal characteristics.

FIG. 10a-e show various views of a single sensor unit 1000. FIG. 10a is a side view of sensor unit 1000 with a top side 1006 and a bottom side 1004. Sensor unit 1000 is connected to output unit via wire 1002. Wire 1002 provides electricity to the sensor 1000 from the output unit, and also transmits the measured sEMG signals from the sensor unit 1000 to the output unit.

FIG. 10b shows a top view of sensor unit 1000. Sensor unit 1000 includes an integrated force sensor 1008. Force sensor 1008 may be, for example, a button that can provide an indication of the applied force when pressed.

FIG. 10c shows a bottom view of sensor unit 1000. Sensor unit 1000 includes a removable electrode pad 1012 that comprises a pair of electrodes 1010. In use, sensor unit 1000 is positioned such that the electrode pair 1010 is in electrical contact with the skin of the subject. Pad 1012 may in an embodiment comprise an adhesive layer for securing the sensor 1000 to the subject and/or a conductive layer on electrode pair 1010 to reduce the electrode-skin contact impedance when in use.

FIG. 10d shows a bottom view of sensor unit 1000 with pad 1012 removed. The sensor unit 1000 includes slots 1014 for inserting the contacts 1016 of the electrode pad.

FIG. 10e shows an example electrode pad 1012. The electrode pad includes electrodes 1010 separated by an inter-electrode distance (IED) measured between the respective centres of the two electrodes 1010, and contacts 1016 for inserting into slots 1014 when mounting pad 1012 into the sensor unit 1004. The contacts 1016 both secure pad 1012 in the sensor unit 1000 and provide a means for transmitting the sEMG signal detected by the electrodes 1010 to the sensor unit 1000 for further processing and/or display by the output unit.

FIG. 10f shows a topside view of electrode pad 1012, including contacts 1016 for inserting into slots 1014 when mounting pad 1012 into the sensor unit 1004 and electrodes 1010.

FIGS. 11a-e show various views of a single sensor unit 2000 in accordance with another embodiment of the technology described herein. FIG. 11a is a top perspective view of sensor unit 2000 with a with a top side 2006 and a bottom side 2004, the bottom side 2004 positioned towards the skin of a subject in use. Sensor unit 2000 is connected to output unit via wire 2002. Wire 2002 provides electricity to the sensor 2000 from the output unit, and also transmits the measured sEMG signals from the sensor unit 2000 to the output unit.

The sensor unit 2000 comprises an integrated force sensor 2008, comprising a button 2017 on the top side 2006 of the sensor unit 2000 to which force is to be applied, the applied force being transmitted to a patient in use when the sensor unit is positioned on the patient's skin. As discussed above, the force sensor will provide an indication of the applied force when pressed. As discussed above, the force sensor may provide a visual feedback such as one or more lights on the sensor unit to indicate that the correct pressure is being applied. Alternatively, visual feedback may be provided via a display, e.g. associated with the output unit. Alternatively (or additionally), other types of feedback could be provided, such as audio and/or haptic feedback.

FIG. 11b shows a bottom view of sensor unit 2000. FIGS. 11c and 11d are exploded perspective views of the sensor unit 2000. FIG. 11e is a cross-sectional view of the sensor unit.

As can be seen from FIG. 11b, the bottom side 2004 of the sensor unit 2000 comprises a pair of connectors 2014 for forming an electrical connection with a pair of electrodes, e.g. in the form of a pair of electrode pads (by connecting to a corresponding mating connectors of each of the electrode pad(s)). Alternatively, a single pad (having two electrical connections) could be attached to the pair of connectors 2014. The electrode pad(s) may be single use and disposable.

The electrode pad(s) adapted to be received by the sensor unit 2000 may be of the types discussed above, e.g. comprising an adhesive layer for securing the electrode pad(s) to the skin of the subject and/or a conductive (e.g. gel) layer to reduce the electrode-skin contact impedance when in use.

FIG. 12 shows an example electrode pad 3000 that may be used in accordance with the technology described herein, for example in combination with the sensor unit 2000. The electrode pad comprises a pair of electrodes 3001 each comprising a respective connector 3004 for forming an electrical connection with the sensor. The connector may be a protruding, mating connector. The electrodes have a centre-to-centre distance (inter-electrode distance, IED).

The electrode pad 3000 of FIG. 12 also comprises a pair of conductive gel layers 3002, each conductive gel layer 3002 contacting a respective (underlying) electrode 3001. In the example shown, each conductive gel layer 3002 has the same size as its respective electrode 3001. As such, the spacing between the electrodes corresponds to the ‘Gel spacing’. Alternatively, the conductive gel layer could be a different size compared to the electrode. The electrode pad 3000 of FIG. 12 also comprises a substrate layer (in this example, an adhesive foam) 3003 which extends transversely beyond the extent of the electrodes 3001 and gel layers 3002, the substrate layer 3003 defining the overall size of the electrode pad (the pad length and pad width).

The example shown in FIG. 12 is an electrode pad comprising a pair of electrodes. As discussed herein, a pair of electrode pads each having a single electrode could be used instead.

Referring back to FIGS. 11a-e, the connectors 2014 of the sensor unit 2000 provide a means for transmitting sEMG signals detected by the electrodes to the sensor unit 2000 for further processing and/or display by the output unit. The connectors 2014 shown are of a mating, ‘snap fit’, connector type. However, other types of connectors or other electrical connection between the sensor unit and the electrodes could be used if desired. For example, instead of providing a mating connector, a conductive adhesive could be used to secure and provide electrical contact with the electrodes or electrode pad(s). In this case, the connector on the electrode (electrode pad) may be flat.

In the embodiment shown in FIGS. 11a-e, the connectors 2014 have a fixed position. However, the connectors could be moveable if desired, e.g. to assist with accommodating different electrode spacings and electrode (or electrode pad) sizes.

The bottom side 2004 of the sensor unit 2000 comprises a recess 2015 for receiving at least part of the pair of electrodes (electrode pad(s)). In the embodiment shown, the connectors 2014 are provided in the recess 2015.

The recess 2015 is adapted (sized, e.g. having a depth D) such that (at least) the electrical connection (interface) to the electrodes (electrode pads) is substantially contained in the recess (e.g. such that connector 3004 shown in FIG. 12 is contained within the recess). In this regard, the recess allows the electrodes (electrode pad(s)) to lie substantially flat, despite any projecting or mating parts of the electrodes which form the electrical connection to the sensor unit 2000. The electrodes (e.g. electrodes 3001 of FIG. 12) may fit transversely within the recess 2015, and may also substantially fit within the depth D of the recess such that the electrodes lie substantially flush with the bottom surface 2018 of the sensor unit. A gel provided with the electrodes (e.g. gel layer 3002) may also fit transversely within, and may be substantially contained within the recess 2015.

The electrode pad(s) (e.g. a substrate layer 3003 of the electrode pad) in an embodiment has a larger size (area) than the recess, such that the electrode pads extend (transversely) beyond the recess and lie against a bottom surface 2018 of the sensor unit. In this regard, the electrode pad(s) may be substantially entirely supported by the bottom surface 2018 of the sensor unit 2000 (such that the electrode pad(s) do not extend beyond the bottom surface 2018 of the sensor unit 2000). This may assist with applying pressure evenly when the button 2017 is pressed. Alternatively, larger electrode pad(s) could be provided which extend beyond the sensor (bottom surface 2018) footprint.

Alternatively, the electrode pad(s) could be completely contained within the recess 2015, e.g. such that the electrode pad(s) lie substantially flush with the bottom surface 2018 of the sensor unit and thus still maintain contact between with a patient's skin.

The provision of the recess 2015 may help prevent uneven pressure forces on the electrodes (electrode pads) which could affect the quality of the sEMG signal obtained by the electrodes. In the case where electrode pad(s) contain (or are provided in combination with) a conductive gel, the recess can also help prevent uneven pressure on the conductive gel, which may otherwise cause gel movement or leakage which could affect sEMG signal quality. To (further) reduce the chance (extent) of gel movement or leakage, a conductive gel of relatively higher viscosity could be used.

Alternatively, a flat electrical connection could be provided between the sensor unit and the electrodes (electrode pads), in which case the recess may be omitted.

As can be seen from FIGS. 11c-e, in this embodiment, the force sensor 2008 comprises the button 2017 in communication with (contacting) a sensor 2009 positioned beneath the button 2017. The button 2017 may comprise an engaging member 2011 that engages with the sensor 2009 and transfers pressure applied to the button to the sensor. The sensor 2009 generates electrical signals in response to (and proportional to) the pressure applied. The electrical signals may be processed by the sensor unit 2000 and/or transmitted to the output unit via the wire 2002.

The sensor 2009 shown is a load cell, which generates an electrical signal in response to load applied to it (as a result of pressure being applied to the button 2017 and transferred to the sensor 2009). The load cell is configured to deform in response to load (pressure) applied, and will return to its original shape when load (pressure) is removed. The load cell in an embodiment comprises a metal body, with one or more integrated strain gauges which produce an electrical signal in response to deformation of the load cell. Alternatively, the load cell could be made of any other suitable and desired material(s). Alternatively, other types of force (pressure) sensor could be used.

The Applicant has found that use of a button 2017 in contact with a load cell provides a relatively robust and reliable mechanism for measuring pressure applied to a patient via the sensor unit 2000. This arrangement is also sensitive to and can measure pressure release. It is also relatively robust during use, and easy to assemble during production of the sensor unit 2000 due to having few moving parts. Alternatively, other force sensor arrangements could be provided, e.g. such as a piezoresistive or piezoelectric force sensor.

To ensure close contact between the button 2017 and sensor 2009, the button 2017 and sensor 2009 may be connected by a non-conductive connection, e.g. joined by a plastic screw). Alternatively, other connection means could be provided, or no connection means could be provided (such that the button simply rests on and/or engages the sensor)

The sensor unit 2000 comprises circuitry (circuits) for processing the sEMG signals received from the electrodes (when attached) and for processing signals from the force sensor 2009. As shown in FIGS. 11c-e, the sensor unit 2000 is provided with a printed circuit board (PCB) 2005 that comprises (has attached to it) various processing elements (circuits) 2007 for processing the signals from the electrodes and force sensor 2009. The electrodes are attached to the PCB 2005 via the connectors 2014, and the force sensor 2009 is also attached to the PCB. In an embodiment, the sensor unit 2000 is configured to performed any required analogue processing of the sEMG signals received from the electrodes (e.g. such as amplification, and applying high and/or low pass filters), and is configured to perform digitisation (analogue-to-digital conversion) of the processed sEMG signals. The sensor unit also performs digitisation of the signals received from the force sensor 2008. The sensor unit 2000 is configured to send the digitised sEMG and force sensor signals to the output unit via the wire 2002.

By attaching the force sensor 2008 and electrodes to the PCB, and providing processing circuits on the PCB, no (or minimal) wiring is needed within the sensor unit 2000 (aside from the wire 2002 which transfers data to the output unit). This reduces the number of possible moving parts within the sensor unit 2000, and provides a robust and easy-to-assemble configuration. Furthermore, avoiding the use of wires and reducing the moving parts within the sensor unit 2000 helps to avoid interference with the sEMG signal from the electrodes, and can therefore help allow small signals of the order of μV (as may be required to be detected for measuring abdominal muscle activity indicative of peritonitis).

Furthermore, by performing the analogue signal processing by the sensor unit 2009 (using circuits provided on the PCB 2005), the processing of signals is done close to the patient. This can also help to improve signal quality and avoid artefacts/interference, as analogue signals are transmitted over only short distances within the sensor unit 2009, and not transmitted outside of the sensor unit to an external processor.

Furthermore, since the wire 2002 is only required to transmit digitised signals (since the circuits of the sensor unit perform analogue-to-digital conversion of sEMG and force sensor signals), it is possible to use a wire configured for transmitting digitised signals, which can be a relatively thin and flexible wire which is not shielded. (In comparison, a thicker, electromagnetically shielded wire may be needed to transmit analogue signals externally from the sensor unit). Where the wire 2002 enters the sensor unit 2000, a strain-relieving component or material may be provided, e.g. such as an adhesive.

In embodiments where plural (e.g. four) sensors are provided for measuring sEMG signals at plural (e.g. four) locations simultaneously (e.g. at four different quadrants of a patient's abdomen), each sensor is may be of the type shown and described above. As such, each sensor may perform its own respective processing of sEMG and pressure sensor signals (e.g. by way of processing circuits provided on a PCB of the sensor unit). Accordingly, the system of plural sensors may be configured to perform distributed processing. The sensors (sensor units 1000, 2000) may each regulate their own timing (have a respective clock). The sensors clocks may be synchronised to a global clock (e.g. maintained by the output unit), by periodically sending a synchronisation signal to the sensor units (from the output unit, via the respective wires 1002, 2002 that connect the sensor units to the output unit). The sensor clocks may be synchronised (by sending a synchronisation signal) before the sensors commence recording sEMG signals for a patient, and then continue using their own respective clocks whilst performing measurements for the patient in question. The sensor clocks may be synchronised (by sending a synchronisation signal) between use on different patients, and/or between different measurements on a same patient.

Synchronising the sensors to the same global clock as described herein has the advantage of allowing data collected at the same time from different sensors to be compared (e.g. by displayed the outputs from the plural sensors simultaneously on a suitable display), which can assist with comparing sEMG activity measured at different locations on a patient to assist with identifying sEMG activity that may indicate health issues such as peritonitis, and to assist with synchronising across all sensor locations the data from any of the interval, breathing, coughing or pressure tests described herein. The synchronisation also allows the time of pressure application and release to be identified relative to all sEMG signals detected by the sensors, to assist with identifying sEMG activity in response to the pressure application at any sensor location (and not just the sensor unit at which the pressure was applied).

Although sending data (e.g. synchronisation signals) to and receiving signals (e.g. digitised sEMG and force sensor signals) from the sensor units 1000, 2000 is done in the above embodiments by using a wire 1002, 2002, in alternative embodiments, data could be sent to (and received from) the sensor units by a wireless communication means (e.g. WiFi, Bluetooth, or other wireless communication means).

In the embodiment shown in FIGS. 11a-e, the sensor unit 2000 is provided with a rigid enclosure, comprising an upper housing component 2001 and a lower housing component 2003, which together enclose at least the force sensor 2009 and PCB 2005. The housing components 2001, 2003 of the sensor unit may be constructed of one or more rigid, non-conducting materials, e.g. such as plastic.

The sensor unit may comprise one or more features to reduce (assist with avoiding) mechanical impact (‘jamming’) and/or resistance (e.g. friction) between moving parts, which might otherwise affect the sEMG signals. For example, the sensor unit may comprise a gap (spacing) between and/or damping for mechanical moving parts. For example, in the embodiment shown in FIG. 11e, the sensor unit comprises a gap between the upper and lower housing components 2001, 2003, in embodiments the gap being maintained when pressure is applied to the (button 2017) of the sensor unit.

In embodiments of the technology described herein (such as shown and described with reference to FIGS. 10a-f and FIGS. 11a-e) a force sensor is incorporated into a sensor unit which is also configured to detect sEMG signals, and in an embodiment a force sensor is incorporated into each of plural sensor units configured to detect sEMG signals.

Incorporating the force sensor into the sensor unit has the advantage that a single unit is required for both applying force to a patient and measuring sEMG signals from a patient, and thus allows the simple placement and use of the system. Furthermore, this means that pressure can be applied and sEMG signals measured at the same area of a patient's body (as such the location at which pressure is applied relative to the sEMG signal measurement is known, compared to e.g. a user applying pressure to some undefined are of the subject).

Furthermore, the user can apply pressure to a subject by pressing the button 2017, and so the user does not need to directly touch the patient. This is advantageous since direct contact with the patient could cause electrical interference with patient and affect the sEMG signals. In embodiments, the housing of the sensor is a non-conductive material, and so provides an electrical isolation barrier, which may assist with reducing electrical interference from a user touching the button 2017. As discussed above, in embodiments, the technology described herein provides a surface electromyography system for medical use, the system comprising a plurality of sensors for detecting muscular electrical activity, and an output unit coupleable to the plurality of sensors and operable to provide outputs representing the outputs of each of the plurality of sensors.

In embodiments, when using the system, a user may be prompted by the system to enter details of a patient for whom electrical muscular activity is to be detected. The patient details may comprise, for example, one or more of (in an embodiment plural of, in an embodiment all of) a patient weight (or BMI), a patient height, and a patient gender. In embodiments, the system may output an indication (suggestion) of an electrode (or electrode pad) size and/or an electrode spacing which should be used for the patient in question. The suggested size and/or spacing may be the same for all sensors of the plurality of sensors, or alternatively may differ for different sensors of the plurality of sensors.

The user may accordingly attach electrodes (electrode pads) (e.g. of the suggested size and/or at the suggested spacing) to the sensors of the plurality of sensors. The system may comprise a storage storing a plurality of electrodes (electrode pads) of different sizes from which the user can choose the desired electrodes. In embodiments, in response to the user attaching electrodes, the system may prompt the user to confirm which electrode sizes have been used. The system may (also) be configured to detect whether (and which) electrodes have been attached to the sensors of the plurality of sensors.

In embodiments (once electrodes have been mounted to the sensors of the plurality of sensors) the sensors can be applied to the skin of a patient by means of an adhesive. In embodiments, the electrodes (electrode pads) comprise a protective film which can be removed to expose a layer of adhesive.

In embodiments, the system is configured to guide the user to one or more (in an embodiment plural) locations to which the sensors should be (simultaneously) applied. In embodiments, the locations comprise locations on four abdominal quadrants of a user. In embodiments, the system may indicate to a user which sensor has been allocated to which location (quadrant), e.g. by displaying an indication on a display.

After guiding the user to place the sensors (and in an embodiment after the user has confirmed via the system that the sensors have been placed), the system may prompt the user to begin a measurement session. The system may be configured to (then) start a measurement session in response to an input from the user, and begin displaying outputs indicative of muscular activity from the plurality of sensors. In an embodiment, the outputs comprise a graph of detected sEMG signals from each sensor against time, in an embodiment displayed simultaneously for each of the sensors. The user may ask a patient to cough, and observe the effect this has on muscular activity by observing the system display. If the display (e.g. graphs) does not look correct, the user may remove and replace an electrode to ensure correct electrode placement.

In embodiments where the sensors each have a respective force sensor, (once a measurement session has commenced) the system may prompt the user to push on the sensors in turn, e.g. pushing the sensors according to a particular order. The system may be configured to indicate to the user when sufficient pressure has been applied to each sensor (e.g. via a signal on the sensor which is being pressed and/or via a signal on a display of the output unit).

In embodiments, the system is configured to display an indication of the force applied to each sensor, in an embodiment simultaneously with the sEMG signals from the sensor, in an embodiment graphing both the sEMG signals and applied force against time. The user may consult the responses graphed to identify signals indicative of peritonitis.

1. A surface electromyography system for medical use, the system comprising:
a plurality of sensors for detecting muscular electrical activity, each sensor of the plurality of sensors comprising a pair of electrodes and operable to provide an output representing the potential difference between the pair of electrodes; and
an output unit coupleable to the plurality of sensors, the output unit operable to provide outputs representing the outputs of each of the plurality of sensors.

2. The surface electromyography system of claim 1, wherein the electrodes of each pair of electrodes of each of the plurality of sensors are separated by a distance within a range of 3 mm and 50 mm, and optionally wherein the distance is within a range of (i) 3 mm and 40 mm, 3 mm and 20 mm, or 3 mm and 5 mm or (ii) 15 mm and 40 mm, 15 mm and 30 mm, or 15 mm and 25 mm; and/or
wherein at least one of the pairs of electrodes comprises circular electrodes with a diameter between 1 mm and 5 mm.

3. The surface electromyography system of claim 1, wherein the plurality of sensors are positionable such that at least one sensor of the plurality of sensors is positioned for measuring electrical activity of an external oblique muscle of a subject, and at least one other sensor of the plurality of sensors is simultaneously positioned for measuring electrical activity of an internal oblique muscle of the subject, and wherein the plurality of sensors comprises at least four sensors.

4. (canceled)

5. The surface electromyography system of claim 1, wherein the pairs of electrodes of the sensors are removable; and/or
wherein each sensor of the plurality of sensors comprises one or more recesses adapted to receive at least part of its pair of electrodes.

6. The surface electromyography system of claim 1, wherein the pairs of electrodes of the sensors comprise electrode pads, the electrode pads being removably mounted in the plurality of sensors; and or/
wherein each sensor comprises a conductive substance for reducing impedance of an electrode to skin contact.

7. The surface electromyography system of claim 1, wherein the system is configured to detect a stimulation applied to a subject during use of the surface electromyography system; and
wherein the system is configured to indicate at least one of an amplitude and a timing of the stimulation.

8. (canceled)

9. The surface electromyography system of claim 7, wherein the stimulation applied to a subject that the system is configured to detect comprises a force applied to a subject during use of the surface electromyography system; and
wherein the system comprises one or more force sensors coupleable to one or more of the plurality of sensors, wherein the one or more force sensors are operable to measure a force applied to the one or more of the plurality of sensors.

10. (canceled)

11. The surface electromyography system of claim 9, wherein each sensor of the plurality of sensors comprises a respective force sensor comprising a force sensitive button

12. (canceled)

13. The surface electromyography system of claim 1, wherein each sensor of the plurality of sensors is configured to perform analogue-to-digital conversion of signals detected by its respective electrode pair, and to transmit the digital signals to the output unit; and
wherein each sensor is configured to perform analogue processing of signals detected by its respective electrode pair prior to performing the analogue-to-digital conversion, the analogue processing comprising at least one of: a first amplification, a low pass filtering, a high pass filtering, a second amplification, and an inversion of a common mode voltage.

14. (canceled)

15. The surface electromyography system of claim 1, wherein each sensor maintains a respective local clock, wherein the local clocks of the sensors are synchronized.

16. The surface electromyography system of claim 1, wherein the system comprises one or more amplifiers for amplifying the output of the one or more of the plurality of sensors, the system configured to amplify the output of each of the sensors to a total gain of at least 1000; and
wherein the one or more amplifiers comprises a first amplifier operable to amplify the output of the one or more of the plurality of sensors and a second amplifier operable to amplify the output of the first amplifier.

17. (canceled)

18. The surface electromyography system of claim 16, the system further comprising:
an electrical circuit operable output a signal comprising an inverted common mode voltage of the system; and
a reference electrode operable to feedback the output of the electrical circuit to the plurality of sensors

19. The surface electromyography system of claim 1, wherein the output unit comprises a display for displaying the outputs representing the outputs of each of the plurality of sensors, preferably wherein the output unit provides an output for each of the sensors that is displayed separately and simultaneously on the display.

20. The surface electromyography system of claim 1, wherein the system comprises one or more high pass filters, the one or more high pass filters operable to attenuate the output of at least one of the plurality of sensors below a cut-off frequency between 10 Hz and 20 Hz; and/or
wherein the system comprises one or more low pass filters, the one or more low pass filters operable to attenuate the output of at least one of the plurality of sensors above a cut-off frequency between 400 Hz and 500 Hz; and/or
wherein the system comprises one or more notch filters for removing power line interference; and/or
wherein the system is operable to apply a wavelet filter to the output of the plurality of sensors; and/or
wherein the system is operable to detect a potential difference between each of the pairs of electrodes of less than 1010 μV, less than 5 μV, less than 3 μV or less than 1 μV.

21. (canceled)

22. The surface electromyography system of claim 1, wherein the plurality of sensors are positionable for measuring muscular electrical activity over a region of the surface of a subject's skin.

23. A plurality of sensors for use in a surface electromyography system for detecting muscular electrical activity, each sensor of the plurality of sensors adapted to receive a respective pair of electrodes and configured to receive signals from its respective pair of electrodes when mounted in the sensor;
each sensor of the plurality of sensors comprising a respective for detector, each sensor configured to receive force signals from its respective force detector simultaneously with receiving signals from its pair of electrodes;
each sensor of the plurality of sensors configured to output signals representative of the signals received from its respective pair of electrodes and force sensor.

24. A method of measuring muscular electrical activity over a region of the surface of a subject's skin using the system of claim 1, the method comprising:
attaching the plurality of sensors to the subject's skin such that the pairs of electrodes are in contact with the subject's skin;
detecting a potential difference between the pairs of electrodes; and
providing an output representative of the potential difference detected by each of the pairs of electrodes.

25. The method of claim 24, further comprising comparing an amplitude of the potential difference detected by the pairs of electrodes to a threshold amplitude.

26. The method of claim 24, further comprising:
applying a force to the subject; and
the system providing an output representative of at least one of the amplitude and the timing of the applied force.

27. The method of claim 24, further comprising:
determining, based on the output representative of the potential difference detected by one or more of the pairs of electrodes, one or more conditions indicative of peritonitis.
